(19) United States
(12) Patent Application Publication
(10) Pub. No.: US 2025/0011878 A1
(43) Pub. Date: Jan. 9, 2025

(54) METHOD TO DETECT RNA BIOMARKERS

(71) Applicant: ITALFARMACO SPA, Milano (IT)

(72) Inventors: Gianluca FOSSATI, Milano (IT); Chiara RIPAMONTI, Monza (IT); Christian STEINKÜHLER, Roma (IT)

(21) Appl. No.: 18/711,937

(22) PCT Filed: Nov. 22, 2022
(86) PCT No.: PCT/EP2022/082762

§ 371 Date: May 21, 2024

(30) Foreign Application Priority Data
Nov. 23, 2021 (IT) 102021000029558

(51) Int. Cl. C12Q 1/6886

(52) U.S. Cl. CPC C12Q 1/6886; C12Q 2600/106; C12Q 2600/158

(57) ABSTRACT

The present invention relates a method based on mRNA biomarkers that can be used to define the efficacious dose and/or the biological activity of inhibitors of histone deacetylase 6 (HDAC6), such as the compound N-hydroxy-4-((5-(thiophen-2-yl)-1H-tetrazol-1-yl)methyl) benzamide, during the clinical treatment of patients affected by cancer. More in particular, the invention refers to the analysis of the gene expression's variation of specific biomarkers in human monocytes, as “gene expression signatures”, in a method to evaluate the clinical efficacy of HDAC6 inhibitors, such as the compound N-hydroxy-4-((5-(thiophen-2-yl)-1H-tetrazol-1-yl)methyl)benzamide.

### BACKGROUND OF THE INVENTION

The genetic material of eukaryotic cells is organized in a complex and dynamic structure called chromatin consisting of DNA and proteins. The main protein components of chromatin are histones, basic proteins which interact with DNA forming the structural unit of chromatin, the nucleosome, the first level of chromosomal compaction within the nucleus. The interaction between basic histone residues and the acidic phosphate backbone of DNA is crucial in determining nucleosome compaction and the accessibility of molecular complexes regulating replication and transcription. This interaction is mainly influenced by multiple post-translational modifications of the N-terminal sequences of core histones such as methylation, phosphorylation, ubiquitination and acetylation. Deacetylation of histone N-terminal lysine residues enables protonation of amine group, which, by carrying a positive charge, interacts with negative charges contained in DNA. Such interaction results in a more compact state of chromatin, leading to silencing of gene expression.

Conversely, acetylation of the same residues prevents ionic bond formation, leading to a less compact form of chromatin which allows greater DNA exposure and the interaction with macromolecular complexes that activate gene transcription.

In addition to these physico-chemical consequences, post-translationally modified residues are also specifically recognized by bromodomain or chromodomain containing “reader” proteins that recognize methylation or acetylation marks and that are involved in the stabilization of repressed or activated chromatin states.

The degree of histone acetylation is regulated by the activity balance of two classes of enzymes: histone acetyl transferases (HAT) and histone deacetylases (HDAC). An alteration of this delicate balance can lead to a loss of cellular homeostasis, commonly found in various human diseases, including cancer, neurological disorders, inflammation, and autoimmune diseases.

Histone deacetylases have been so classified as they catalyse the deacetylation of amine groups of histone N-terminus lysine residues. This enzymatic activity on lysine histone tails, further classified them as “erasers” as opposed to HAT enzymes called “writers”. Subsequently, it has been found that there is a large number of substrates of these enzymes as their activity is also exerted on non-histone proteins substrates of HAT enzymes, containing N-acetyl-lysine. These substrates comprise transcription factors, DNA repair enzymes and many other nuclear and cytoplasmic proteins.

The human HDAC family consists of 18 enzymes, divided into two groups: zinc-dependent HDACs and NAD-dependent HDAC, also known as sirtuins (class III). Zinc-dependent HDACs are further distributed into four classes: 1) Class I, including HDAC1, 2, 3 and 8, ubiquitous isoenzymes mainly located in the nucleus; 2) Class IIa, including HDAC4, 5, 7 and 9, isoenzymes located both in the nucleus and the cytoplasm; 3) Class IIb, including HDAC6 and HDAC10, mainly located in the cytoplasm and 4) Class IV, including only HDAC11. Unlike Class I HDACs, Class IIa and to a certain extent IIb have a tissue-specific expression.

By regulating gene expression and acting on histones and transcription factors, it is clear that these enzymes are involved in a myriad of cellular functions. In addition, by acting on numerous protein substrates, these enzymes, are involved in many other processes, such as signal transduction and cytoskeleton rearrangement.

Several HDAC inhibitors have been developed over the last 20 years and 5 molecules have been approved for the treatment of cancer in humans (Vorinostat, Romidepsin, Belinostat, Panobinostat and Chidamide). All these molecules inhibit multiple HDAC subtypes that also play a role in normal tissues. Their therapeutic potential is therefore limited by toxicities such as thrombocytopenia, GI tract toxicity or fatigue.

The attention of the scientific community has thus focused on the synthesis and study of selective inhibitors for individual HDAC isoforms, aiming to develop molecules with better pharmacological capabilities.

Selective inhibitors for a specific HDAC isoform, especially HDAC6, may be particularly useful for treating pathologies related to proliferative disorders and protein accumulation, immune system disorders, respiratory, neurological and neurodegenerative diseases, such as stroke, Huntington's disease, ALS and Alzheimer's disease. HDAC6 is a member of the Zn-dependent histone deacetylase family with some unique and distinguishing features, such as the presence of two active sites with different catalytic activities and, probably, different biological roles. HDAC6 substrates include α-tubulin, Hsp90 (Heat Shock Protein 90), cortactin, β-catenin.

Modulation of the acetylation status of these proteins by HDAC6 has been correlated with several important processes, such as immune response (Wang et al., Nat. Rev. Drug Disc. (2009), 8(12), 969-981; Kalin J H et al. J. Med. Chem. (2012), 55, 639-651; de Zoeten E F et al. Mol. Cell. Biol. (2011), 31(10), 2066-2078), regulation of microtubule dynamics, including cell migration and cell-cell interaction (Aldana-Masangkay et al., J. Biomed. Biotechnol. (2011), ID 875824), and degradation of misfolded protein. HDAC6 is constitutively expressed in most of the body tissues and has a prevalent cytosolic localization although it also exerts an activity in the nuclear compartment. HDAC6 activities are altered in pathologies such as cancer, neuropathies, respiratory diseases and autoimmune pathologies (Li, T., Zhang, C., Hassan, S., Liu, X., Song, F., Chen, K., Zhang, W., and Yang, J. (2018). Histone deacetylase 6 in cancer. Journal of Hematology & Oncology 11, 111); Prior, R., Van Helleputte, L., Klingl, Y. E., and Van Den Bosch, L. (2018). HDAC6 as a potential therapeutic target for peripheral nerve disorders. Expert Opinion on Therapeutic Targets 22, 993-1007).

In the context of cancer, HDAC6 has been recognized as a key modulator of the function of the tumor microenvironment, where it controls anti-tumor immune responses by regulating the expression of PD-L1 in immune cells and in tumor cells. HDAC6 is also involved in regulating expression of oncoproteins, especially in hematologic tumours, such as various types of leukaemia (Fiskus et al., Blood (2008), 112(7), 2896-2905; Rodriguez-Gonzales, Blood (2008), 112(11), abstract 1923) and multiple myeloma (Hideshima et al., Proc. Natl. Acad. Sci. USA (2005), 102(24), 8567-8572). Regulation of α-tubulin acetylation by HDAC6 may be implicated in metastasis onset, wherein cellular motility plays an important role (Sakamoto et al., J. Biomed. Biotechnol. (2011), 875824).

Further, in stark contrast to the preclinical and clinical observations made with non-selective HDAC inhibitors, that suffer from dose-limiting toxicity, HDAC6 inhibitors do not show any evident signs of toxicity. Also HDAC6 knock out mice are viable, develop normally and have no evident signs of pathologic alterations. This is in contrast to what is observed upon ablation of the expression of other HDAC subtypes.

In conclusion, selective inhibitors of HDAC6 hold the promise of bearing a considerable therapeutic potential while being very well tolerated.

In WO2018/189340 we disclosed a particularly effective HDAC6 inhibitor, N-hydroxy-4-((5-(thiophen-2-yl)-1 H-tetrazol-1-yl) methyl) benzamide, that has been found strikingly active in modulating antitumor immune responses. In keeping with the known excellent tolerability of HDAC6 inhibitors, this molecule was well tolerated in rats, mice and dogs (1000 mg/kg), suggesting that it will be well tolerated also in humans.

This hypothesis is further supported by the clinical data obtained with the HDAC6 inhibitors Ricolinostat and KA2507, that were shown to be very well tolerated in patients (Amengual J E et al. Oncologist (2021) 3:184-e366; Tsimberidou A M et al. Clin Cancer Res (2021) 27:3584-3594).

While being highly desirable for a drug, the lack of toxic side effects poses however challenges to its clinical development.

Classically, oncology phase I clinical protocols plan a dose escalation until the maximum tolerated dose is reached. Cohorts are usually expanded at this dose level and a recommended phase II dose is derived from information on tolerability, PK and initial signs of efficacy. In the absence of any toxic side effects, other parameters need to be used in order to define a dose.

Biomarkers can be very helpful in defining a biologically effective dose.

US2012/176076 discloses a kit for determining the treatment efficacy of a histone deacetylase 6 inhibitor (HDAC6) in a subject having multiple myeloma, comprising a detection agent that binds specifically to a HDAC6 biomarker RNA, selected from a miRNA (SEQ ID N. 1-23), a mRNA (SEQ ID N. 24-25) and a small non coding RNA (SEQ ID N. 26-27). The only mRNA sequences that encode for a protein are SEQ ID N. 24, that corresponds to the Homo sapiens hypoxia inducible factor 1 subunit alpha (HIF1A), transcript variant 2 mRNA and SEQ ID N. 25, that corresponds to Homo sapiens protein tyrosine phosphatase receptor type U (PTPRU), transcript variant 2 mRNA.

For HDAC6 inhibitors, the increase in tubulin acetylation levels can be used as a readout. For example, patients receiving a dose of an HDAC6 inhibitor will undergo blood draws at different time points after administration of the drug and acetyl tubulin can be determined in PBMCs using western blot analysis. While relatively straightforward, this method is qualitative or semi-quantitative and measures a pharmaco-dynamic marker that has no direct relationship with the antitumor activity.

There is therefore the need of a quantitative method based on biological markers evaluable in patients samples, that can be used to assess the pharmacologically active dose of an HDAC6 inhibitor and more particularly of the compound N-hydroxy-4-((5-(thiophen-2-yl)-1 H-tetrazol-1-yl)methyl) benzamide.

### Definitions

Unless otherwise defined, all terms of art, notations and other scientific terminology used herein are intended to have the meanings commonly understood by those person's skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The terms “comprising”, “having”, “including” and “containing” are to be understood as open terms (meaning “including, but not limited to”) and are to be considered as a support also for terms such as “essentially consist of”, “essentially consisting of”, “consist of” or “consisting of”.

The terms “essentially consists of”, “essentially consisting of” are to be understood as semi-closed terms, meanings that no other ingredient affecting the novel characteristics of the invention is included (therefore optional excipients can be included).

The terms “consists of”, “consisting of” are to be understood as closed terms.

The term “gene expression signature” herein refers to an expression pattern derived from combination of several mRNA or RNA (i.e. transcripts) used as biomarkers.

According to the present invention the term “expression level of a RNA biomarker” refers to detecting and/or quantifying the RNA or mRNA of a specific gene (biomarker), such as determining and/or quantifying the over-expression or the under-expression of the RNA or mRNA as compared to a control; determining the presence or absence of an RNA or mRNA in a sample; determining the sequence of the RNA; determining any modifications of the RNA, or detecting any mutations or variations of the RNA. The RNA level may be determined to be present or absent, greater than or less than a control, or given a numerical value for the amount of RNA, such as the copies of RNA per microliter. The expression level of an RNA can be quantified, by absolute or relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between treatment/no treatment to quantify the changes in hybridization intensity and, by implication, in transcription level.

The term “biomarkers” (short for biological markers) herein refers to biological indicators (for example a transcript, i.e. mRNA) and/or measures of some biological state or condition.

According to the present invention a patient affected by cancer is “responsive” to a therapeutic agent if the growth rate of the tumor size or of the cancer is inhibited as a result of contact with the therapeutic agent, compared to the growth in the absence of contact with the therapeutic agent. The growth of a cancer can be measured in a variety of ways. For instance, the size of a tumor or measuring the expression of tumor markers appropriate for that tumor type.

A patient affected by cancer is “non-responsive” to a therapeutic agent if the growth rate of the tumor size or of the cancer is not inhibited, or inhibited to a very low degree, as a result of contact with the therapeutic agent when compared to the growth in the absence of contact with the therapeutic agent. As stated above, growth of a cancer can be measured in a variety of ways, for instance, the size of a tumor or measuring the expression of tumor markers appropriate for that tumor type.

The feature of being non-responsive to a therapeutic agent is a highly variable one, with different cancers exhibiting different levels of “non-responsiveness” to a given therapeutic agent, under different conditions.

Still further, measures of non-responsiveness can be assessed using additional criteria beyond growth size of a tumor such as, but not limited to, patient quality of life and degree of metastases.

According to the present invention the terms “up-regulation”, “up-regulated”, “over-expression” and any variations thereof are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

According to the present invention the terms “down-regulation”, “down-regulated”, “under-expression”, “under-expressed” and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

According to the present invention the term “biological activity” of the HDAC6 inhibitor refers to the modulation/variation of the expression of any genes described in the present invention.

According to the present invention the term “efficacious dose” refers to the dosage of the HDAC6 inhibitor that can be considered effective for the treatment of cancer in a patient.

### FIGURES

FIG. 1. Scheme of the experimental procedures carried out to obtained the gene expression data by RNAseq.

FIG. 2. Sample to sample distance analysis (2A) and principal component analysis (2B).

FIGS. 3A and 3B. The Venn diagram shows the number of genes that are selectively or commonly upregulated by a specific treatment. Generated by the tool of Oliveros, J. C. (2007-2015) Venny, an interactive tool for comparing lists with Venn's diagrams. htts://ioinfog.cnb.csic.es/tools/venny/index.html.

FIG. 4. Purified human monocytes were treated as indicate and the expression of CD274/PD-L1 was analyzed from RNAseq data.

FIGS. 5A, 5B and 5C. Human monocytes downregulates the expression of CD84 upon ITF3756 treatment (A). CD84 Schematic structure (B) and immune cells expression (C).

FIG. 6. ITF3756 induces a strong downregulation of RANK/TNFRSF11a to a similar extent of TNF-α.

FIGS. 7A and 7B. ITF3756 reduced the expression of CXCL2 (A) and CXCL3 (B) in human monocytes.

FIG. 8. ITF3756 strongly down-modulates the expression of STAB1 gene, alone and in combination with TNF-α.

FIG. 9. ITF3756 increases the expression of NBEAL2, alone and in combination with TNF-α.

FIG. 10. ITF3756 upregulates the expression of LTBP4.

FIG. 11. Shows the effect of ITF3756 on the expression of FATP1/SLC27A1 and the opposed effect of ITF3756 and TNF-α on the expression of FATP1/SLC27A1 in human monocytes.

FIG. 12. Shows that only ITF3756 induced a modulation of IRF6 expression while TNF-α or the combination had no effect.

FIG. 13. ITF3756 upregulates ANXA6 gene expression alone and in an apparently synergistic way in the presence of TNF-α.

FIG. 14. ITF3756 upregulates CD40 gene expression and when combined with TNF-α slightly increases the upregulation of CD40 expression induced by TNF-α alone.

FIG. 15. The M2 macrophage marker CD163 is down-modulated by ITF3756, alone and in the presence of TNF-α.

FIG. 16. The M2 macrophage marker CD204/MSR1 is down-modulated by ITF3756, alone and strongly down-modulated in the presence of TNF-α.

FIG. 17. The M2 macrophage marker CD206/MRC1 is strongly down-modulated by ITF3756, alone and in the presence of TNF-α.

FIG. 18. The M2 macrophage marker MMP9 is down-modulated by ITF3756. No modulation compared to untreated control was observed when cells were treated with TNF-α or ITF3756+TNF-α.

FIG. 19. The M2 macrophage marker ADA is downmodulated by ITF3756. TNF-α and the combination induces an upregulation of ADA gene expression.

FIGS. 20A and 20B. shows the modulation of STAB1 and IRF6 genes in PBMC treated with 1 μM ITF3756. A. downmodulation of STAB1. B. upregulation of IRF6.

FIGS. 21A and 21B. Shows that ITF3756 induces persistent tubulin acetylation in vivo. A. Western blots showing acetyl tubulin and tubulin bands. B. Bands intensity were quantified to calculate the fold increase of tubulin acetylation normalized to total tubulin content.

FIGS. 22A, B, C, D and E. Tumor bearing animals responds differently to ITF3756 treatment. Animals treated with ITF3756 can be classified in three different groups. A. ITF3756 groups compared to vehicle group (mean value of tumor volume±SEM). Panels B. C. D and E illustrate the variation of tumor volume for single animal during the study in the four groups indicated.

FIGS. 23A, B, C, D and E. Tumor bearing animals responds differently to anti PD-1 treatment. Animals treated with anti-PD-1 can be classified in three different groups. A, anti PD-1 groups compared to vehicle and isotype control groups (mean value of tumor volume±SEM). Panels B, C, D, E, illustrate the variation of tumor volume for single animal during the study in the four groups indicated.

FIGS. 24A, B and C. MMP9 downregulation in the tumor microenvironment correlates with responders mice. CT26 bearing mice treated with ITF3756 have a statistically significant reduction of MMP9 expression compared to non responders (B). This correlation is not present if all animals are considered (A) or if anti PD-1 treated animals are considered (C).

FIGS. 25A, B and C. IRF6 upregulation in the tumor microenvironment correlates with responders mice. Responder mice treated with ITF3756 have a striking expression upregulation of IRF6 compared to non responders (B). This correlation is not present if all animals are considered (A) or if anti PD-1 treated animals are considered (C).

FIGS. 26A, B and C. Upregulation of CD40 in the tumor microenvironment correlates with responder mice. Responder mice treated with ITF3756 have a statistically significant upregulation of CD40 expression compared to non-responder mice (B). This correlation is not present if all animals are considered (A) or if anti PD-1 treated animals are considered (C).

### DISCLOSURE OF THE INVENTION

We have surprisingly found that our selective HDAC6 inhibitor N-hydroxy-4-((5-(thiophen-2-yl)-1 H-tetrazol-1-yl)methyl)benzamide (also called ITF3756), can up- and/or down-modulate the expression of different genes in myeloid cells activated by pro-inflammatory stimuli, and that it can have a broader effect on gene expression on both unstimulated and TNF-α treated human monocytes.

In particular, it has been observed that human monocytes stimulated with TNF-α strongly upregulated the PD-L1 gene and that the surface expression of PD-L1 and these upregulations are inhibited by ITF3756. Furthermore, the data in-vitro reported in the experimental section demonstrates that the treatment of human monocytes with ITF3756 up-regulates the expression of NBEAL2, FATP1/SCL27A1, LTBP4, CD40, ANXA6 and IRF6 genes and down-regulated the expression of CD84, CD276, RANK/TNFRSF11a, CXCL2, CXCL3, CD163, CD204/MSR1, CD206/MRC1, ADA, MMP9 and STAB1 genes.

The results obtained by the inventors led therefore to the identification of a panel of genes, also called “gene expression signature”, that can be directly modulated by HDAC6 inhibitors, and more specifically by the molecule ITF3756, and that can be used as a gene expression signature with paramount importance for the clinical development of this class of molecules.

Furthermore, said data have been confirmed also from the experimental data obtained in-vivo, wherein it has been observed that in CT26-bearing animals there is a significant downregulation of MMP9 gene and an upregulation of IRF6 and CD40 genes that correlate with responder animals of ITF3656 treated group, demonstrating that the expression level of said genes is strictly related to the effect of the HDAC6's inhibitor in the treated animals.

An embodiment of the present invention is therefore a method for evaluating the efficacious dose and/or the biological activity of a HDAC6 inhibitor, comprising the step of:
a) determining the expression level of at least one RNA biomarker modulated by a HDAC6 inhibitor and selected from CD84, RANK/TNFRSF11a, CXCL3, CXCL2, STAB1, CD163, CD204/MSR1, CD206/MRC1, MMP9, NBEAL2, LTBP4, ANXA6, FATP1/SLC27a1, ADA, CD276, CD40 or IRF6 genes in a biological sample;
b) comparing said expression level with that of a reference sample.

According to a preferred embodiment, said method evaluate the efficacious dose and/or the biological activity of a HDAC6 inhibitor during the clinical treatment of a patient affected by cancer.

In a further preferred embodiment said method evaluate the efficacious dose and/or the biological activity of a HDAC6 inhibitor after the clinical treatment of a patient affected by cancer. This can be useful to evaluate the condition of the patient after the treatment.

According to a further preferred embodiment, said method is an in vitro or ex-vivo method.

Preferably, said reference sample derives from a healthy subject specimen not treated with a HDAC inhibitor, from a subject affected by cancer not treated with a HDAC inhibitor or from a subject at the beginning of the treatment (t=0) with a HDAC inhibitor.

According to a preferred embodiment, said HDAC inhibitor is selected from tubacin, tubastatin, nexturastat, ACY-1215, ACY-738, ACY-1083, KA2507, T518, SW100 or N-hydroxy-4-((5-(thiophen-2-yl)-1 H-tetrazol-1-yl)methyl) benzamide (ITF3756), preferably said HDAC inhibitor is the compound N-hydroxy-4-((5-(thiophen-2-yl)-1 H-tetrazol-1-yl)methyl) benzamide.

According to a preferred embodiment, the method of the present invention further comprises the step c) classifying the subjects as responsive or non-responsive to a clinical treatment.

Preferably, the patients affected by cancer are classified as responsive or non-responsive to the clinical treatment, based on whether the expression value of the at least one RNA biomarker is above or below the threshold expression value.

In one exemplary embodiment, a sample expression value greater than the threshold expression value indicates a patient that will be responsive to the anti-cancer therapeutic treatment. In another exemplary embodiment, a sample expression value below the threshold expression value indicates a patient that will not be responsive to an anti-cancer treatment.

In another exemplary embodiment, a sample expression value greater than the threshold expression value indicates a patient that will be non-responsive to the anti-cancer therapeutic treatment. In another exemplary embodiment, a sample expression value below the threshold expression value indicates a patient that will be responsive to an anti-cancer treatment. It is for example demonstrated in the examples, that the down-regulation of MMP9 and upregulation of IRF6 and CD40 correlate with responder animals.

In further exemplary embodiment, a sample expression value below the threshold expression value indicates the patient has a cancer type, or is at risk of developing a cancer type or that is not responsive to the HDAC6 inhibitor. In another exemplary embodiment, a sample expression value above the threshold expression value indicates the patient has a cancer type, or is at risk of developing a cancer type or that is responsive to HDAC6 inhibitor. In another example embodiment, a sample expression score above the threshold score indicates the patient has a cancer sub-type with a good clinical prognosis. In another example embodiment, a sample expression score below the threshold score indicates a patient with a cancer subtype with a poor clinical prognosis.

According to a further preferred embodiment, in the method of the present invention the expression level of the biomarkers NBEAL2, LTBP4, ANXA6, FATP1/SCL27a1, CD40 or IRF6 is up-regulated by a HDAC6 inhibitor and the expression level of the RNA biomarkers CD84, RANK/TNFRSF11a, CXCL3, CXCL2, STAB1, CD163, CD204/MSR1, CD206/MRC1, ADA, CD276 or MMP9 is down-regulated by a HDAC6 inhibitor.

According to a preferred embodiment, in the method of the present invention the expression level of at least the RNA biomarkers STAB1, CD84, CD206/MRC1, MMP9, CD163, CD40 and IRF6 is evaluated, preferably the expression level of at least the RNA biomarkers MMP9, CD40 and IRF6 is evaluated.

According to a further preferred embodiment, said cancer is selected from Adrenocortical Carcinoma, Anal Cancer, Astrocytomas, Basal Cell Carcinoma of the Skin, Bladder Cancer, Brain Tumors, Breast Cancer, Carcinoma of Unknown Primary, Cardiac Tumors, Cervical Cancer, Cholangiocarcinoma, Colorectal Cancer, Endometrial Cancer, Esophageal Cancer, Intraocular Melanoma, Fallopian Tube Cancer, Gallbladder Cancer, Gastric Cancer, Gastrointestinal Carcinoid Tumor, Gastrointestinal Stromal Tumors (GIST), Germ Cell Tumors, Testicular Cancer, Head and Neck Cancer, Hepatocellular Carcinoma, Islet Cell Tumors, Pancreatic Neuroendocrine Tumors, Langerhans Cell Histiocytosis, Leukemias, Lung Cancer (Non-Small Cell, Small Cell, Pleuropulmonary Blastoma, and Tracheobronchial Tumor), Melanoma, Merkel Cell Carcinoma, Mesothelioma, Midline Tract Carcinoma With NUT Gene Changes, Multiple Endocrine Neoplasia Syndromes, Multiple Myeloma/Plasma Cell Neoplasms, Myelodysplastic Syndromes, Myelodysplastic/Myeloproliferative Neoplasms, Neuroblastoma, Ovarian Cancer, Pancreatic Cancer, Paraganglioma, Parathyroid Cancer, Penile Cancer, Pheochromocytoma, Pituitary Tumor, Primary Peritoneal Cancer, Prostate Cancer, Renal Cell Cancer, Retinoblastoma, Sarcomas, Squamous Cell Carcinoma of the Skin, Thymoma and Thymic Carcinoma, Thyroid Cancer, Transitional Cell Cancer of the Renal Pelvis and Ureter, Uterine Cancer, Vaginal Cancer, Vascular Tumors, Vulvar Cancer and Wilms Tumor.

Preferably, said cancer is selected from melanoma, renal cell carcinoma, non small cell lung cancer and colorectal cancer.

Preferably, the biological sample is a tissue sample or a body fluid, preferably said tissue sample is a tumor biopsy or blood cells; preferably said body fluid is blood, serum or plasma.

More preferably said blood cells are monocytes, or peripheral blood mononuclear cells (PBMC).

According to the present invention, said monocytes can be isolated from patients affected by cancer or by in-vitro plates wherein the cell cultures of monocytes have been treated with a HDAC6 inhibitor.

According to a preferred embodiment, the biomarker is an RNA transcript. As used herein “RNA transcript” refers to both coding and non-coding RNA, including messenger RNAs (mRNA), alternatively spliced mRNAs, ribosomal RNA (rRNA), transfer RNA (tRNA), small nuclear RNAs (snRNA), and antisense RNA. Measuring mRNA in a biological sample may be used as a surrogate for detection of the level of the corresponding protein and gene in the biological sample. Thus, any of the biomarkers or biomarker panels described herein can also be detected by detecting the appropriate RNA.

According to a preferred embodiment, the expression level of the biomarkers in step a) is detected by RNA sequencing, quantitative RT-PCR (qRT-PCR), digital PCR, Affymetrix microarray, custom microarray or nanostring technology.

Methods of biomarker expression profiling include, but are not limited to quantitative PCR, NGS, northern blots, southern blots, microarrays, SAGE, or other technologies that can measure the RNA, mRNA or protein level of a specific biomarker.

The overall expression data for a given sample may be normalized using methods known to those skilled in the art in order to correct for differing amounts of starting material, varying efficiencies of the extraction and amplification reactions.

According to a preferred embodiment, the step a) of the method of the present invention is made of the following sub-steps:
(i) isolating PBMC and/or monocytes;
(ii) extracting RNA samples;
(iii) quantifying at least one RNA biomarker selected from CD84, RANK/TNFRSF11a, CXCL3, CXCL2, STAB1, CD163, CD204/MSR1, CD40, CD206/MCR1, MMP9, NBEAL2, LTBP4, ANXA6, FATP1/SLC27a1, ADA, CD276 and IRF6 genes;
(iv) evaluating the differently expressed biomarkers by bioinformatics analysis of the RNAs biomarker quantified.
(v) correlating the results obtained from point (iv) with responders or non responder patients.

A further embodiment of the present invention is a kit for evaluating the efficacious dose and/or the biological activity of a HDAC6 inhibitor, comprising a multi-well plate and suitable primers and/or probes for determining the expression level each of the RNA biomarkers to be detected.

Preferably said RNA biomarker are selected from CD84, RANK/TNFRSF11a, CXCL3, CXCL2, STAB1, CD163, CD204/MSR1, CD40, CD206/MCR1, MMP9, NBEAL2, LTBP4, ANXA6, FATP1/SLC27a1, ADA, CD276 and IRF6 genes.

A further preferred embodiment is a kit for use in evaluating the efficacious dose and/or the biological activity of a HDCA6 inhibitor, comprising a multi-well plate and suitable primers and/or probes for determining the expression level of at least one of the RNA biomarkers selected from CD84, RANK/TNFRSF11a, CXCL3, CXCL2, STAB1, CD163, CD204/MSR1, CD40, CD206/MCR1, MMP9, NBEAL2, LTBP4, ANXA6, FATP1/SLC27a1, ADA, CD276 and IRF6 genes.

### EXAMPLES

### Materials and Methods

### Monocytes Purification and TNF-α Activation

PBMCs used for the experiments were obtained from Buffy Coats of healthy donors (all samples tested negative for transmissible diseases as required for blood transfusion) separated over a Ficoll-Hypaque gradient (Biochrom).

Monocytes were purified by negative selection from 100×10⁶ PBMC using Pan Monocytes Isolation Kit (Milteny) following manufacturer's instructions. By Pan Monocyte Isolation kit untouched monocytes are isolated from human PBMCs and the simultaneous enrichment of classical (CD14⁺⁺CD16⁻), non classical (CD14⁺CD16⁺⁺) and intermediate (CD14⁺⁺CD16⁺) monocytes is achieved. Non-monocytes, such as T cells, NK cells, B cells, dendritic cells, and basophils are indirectly magnetically labeled using a cocktail of biotin-conjugated antibodies and anti-Biotin MicroBeads. Briefly, 400 μL of Buffer (PBS 1×, 0.5% BSA and 2 mM EDTA), 100 μL of FcR Blocking Reagent and 100 μL of Biotin-Antibody Cocktail were added to PBMC, previously washed with PBS by centrifugation 300×g for 5 minutes, mixed and incubate for 5 minutes in the refrigerator (2-8° C.). After incubation, 300 μL of Buffer and 200 μL of anti-biotin microbeads were added to the cells, mixed and incubate further for 10 minutes in the refrigerator. After incubation cells were processed by subsequent magnetic cell separation. Cell suspension was applied onto the column and the flow-through containing unlabeled cells, representing the enriched monocytes, was collected. Purified monocytes were washed with Buffer by centrifugation 300×g for 5 minutes and counted in PBS. Purified monocytes (1,0×10⁶/ml) were pre-treated for 2 h with ITF3756 1 μM or DMSO (0.005%) in 12-well plates in 1 ml final volume of complete medium (RPMI (Biochrom), FCS 10% and penicillin/streptomycin 1× (Sigma)). The cells were then stimulated or not with TNF-α (100 ng/mL, Peprotech) for 4h. After incubation with ITF3756 and TNF-α, the cells were collected, washed with PBS by centrifugation 300×g for 5 minutes and stored at −80° C.

Samples were thawed on the bench for 2 minutes and total RNA was extracted with Trizol reagent (Thermo Fisher Scientific), following manufacturer's instructions. Briefly, Trizol reagent (0.75 ml Trizol per 5-10×106 cells) was added to the samples and the samples were incubated at room temperature (RT) for 5 minutes. After incubation, chloroform (150 μL per 0.75 mL Trizol) was added to the samples and after 3 minutes of incubation at RT, samples were centrifuged for 12000×g for 14 minutes at 4° C. After centrifugation, the mixture separates into 3 phases and RNA remains in the aqueous phase. The aqueous phase was removed for each sample and placed in new tubes, and isopropanol (0,375 ml per 0.75 ml of Trizol) was added in each tube. Samples was incubated for 30 minutes in the refrigerator (2-8° C.). After incubation, samples were centrifugated for 12000×g for 10 minutes at 4° C., the supernatant was removed and 75% ethanol (0.75 ml per 0.75 ml of Trizol) was added to samples. Samples were centrifuged for 7500×g for 5 minutes at 4° C., and the supernatant discarded. RNA was air dried on the bench for 5-10 minutes. RNA Samples was then resuspended in water (10-50 μL).

RNA concentration was determined by measuring the absorbance at a of 260 nm with a NanoDrop 1000 spectrophotometer (Thermo Scientific). By also measuring the absorbance at 280 it is also possible to estimate the degree of RNA contamination. The 260 nm/280 nm absorbance ratio allows for the identification of protein contamination. The sample was considered sufficiently pure if the 260 nm/280 nm absorbance ratio is approximately 2. The integrity of RNA extracted was assessed by capillary electrophoresis using the Agilent 2100 Bioanalyzer instrument (Agilent Technologies) with the Agilent RNA 6000 Pico kit (Agilent Technologies). The system allows the simultaneous analysis of up to 12 samples using a high purity RNA ladder with a known concentration as a reference. The protocol includes a denaturation step, for 2 minutes at 70° C., of all the samples and RNA ladder and a step for preparing the chip with the run gel containing a fluorescent intercalator. The RNA molecules bind the intercalating molecule and the fluorescence of the molecules separated by electrophoresis is detected by the instrument.

The gel was prepared by pipetting 550 μL of RNA gel matrix into a spin filter (provide by the kit). After centrifugation at 1500×g for 10 min at RT, 65 μL aliquots of filtered gel were prepared. RNA dye concentrate was equilibrated at RT for 30 minutes, then vortexed, spinned down and 1 μL of dye was added into a 65 μL aliquot of filtered gel. The gel-dye mix were mixed and centrifugated at 13000×g for 10 minutes RT. New RNA chip was putted on the chip priming station and 9 μL of gel-dye mix was pipetted in the assigned well and distribute by plunger into the chip. 5 μL of RNA marker was added in all 11 sample wells and in the ladder well. Then, 1 μL of ladder in the ladder well and 1 μL of sample in each of the 11 sample wells was added to the chip. The chip was vortex for 1 minute at 2400 rpm and run in the Agilent 2100 Bioanalyzer instrument within 5 min.

The software allows to obtain for each sample an estimate of the degree of purity by evaluating the RNA Quantity Index (RQI), calculated on the basis of an algorithm that assigns a value from 1 to 10 to each sample as a function of the rRNA 28S/rRNA 18S ratio. The sample is considered to have a high degree of purity if this index is greater than or equal to 7.5.

### RNAseq

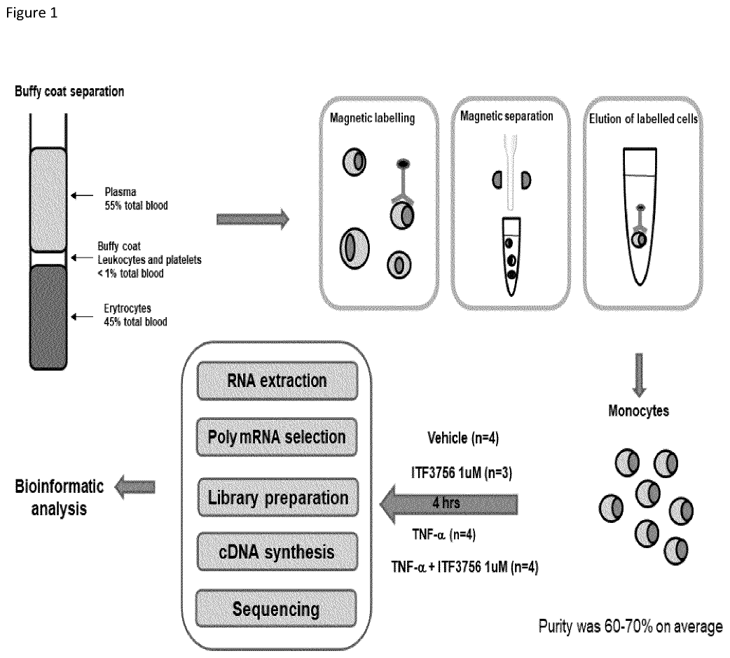

The scheme of the experimental procedures carried out to generate the RNA sequence data is represented in FIG. 1.

### RNA Extraction

To determine the quantity and the integrity/purity of RNA samples, check controls were first performed by the Agilent Technologies 2100 Bioanalyzer using RNA 6000 LabChip® kit (Agilent #5067-1511). The Bioanalyzer is a bio-analytical device based on a combination of microfluidic chips, voltage-induced size separation in gel filled channels and laser-induced fluorescence (LIF) detection on a miniaturized scale. The RNA Integrity Number (RIN) software algorithm allows the classification of total RNA, based on a numbering system from 1 to 10, (with 1 being the most degraded and 10 being the most intact). All the samples with a RIN below 7.5 were discarded while the others were processed for libraries preparation and sequencing.

PolyA mRNA Selection

mRNA was isolated from 200 ng of total RNA using poly-T oligo-attached magnetic beads using two rounds of purification (positive selection) as suggested in the TruSeq RNA Sample Preparation manual (Illumina #15015050).

Library Preparation and cDNA Synthesis

Purified samples were processed using TruSeq RNA-Seq v2 Library Preparation Kit. Shortly, chemical fragmentation was carried out using divalent cations under elevated temperature in Illumina proprietary fragmentation buffer. First strand cDNA was synthesized using random oligonucleotides and SuperScript II (Invitrogen #18064-014). Second strand was subsequently performed using DNA Polymerase I and RNase H. After Agencourt AMPure XP beads purification (Beckman #A63882) which allows size selection of fragments, the overhangs were converted into blunt ends via exonuclease/polymerase activities, then enzymes were removed. DNA fragments were adenylated in their of 3′ends, then Illumina TruSeq PE adapter indexed oligonucleotides were ligated, double purified and selectively enriched using Illumina PCR primer cocktail in a PCR reaction. PCR library products were purified with AMPure XP beads, quality checked using the Agilent DNA 1000 assay (Agilent #5067-1504) on a Agilent Technologies 2100 Bioanalyzer and quantified using Qubit 2.0 Fluorometer with dsDNA Broad Range Assay kit (Thermo Fisher Scientific #Q32850). The indexed individual libraries were pooled to obtain equimolar concentrations for each sample, and then processed for cluster generation.

### Sequencing

Pooled libraries were loaded on a Single End Flow Cell using the cBot System (Illumina) and the TruSeq PE Cluster Generation kit v3 (Illumina #PE-401-3001). The TruSeq technology supports massively parallel sequencing using a proprietary reversible terminator-based method 5 (1 Mar. 2021), that enables detection of single bases as they are incorporated into growing DNA strands. At the end of the run, ˜30M of 75 bp single-end reads were generated on a HiSeq2500 instrument (Illumina) using TruSeq SBS v3 reagents (Illumina #FC-401-3001). Finally, demultiplexed FASTQ files were generated according to the Illumina Pipeline data analysis.

### Bioinformatic Analysis

Low quality ends and adapters were trimmed from single-end reads using TrimGalore http://www.bioinformatics.babraham.ac.uk/projects/trim_galore/. Transcript abundance was estimated with Kallisto (Bray et al, 2016) and differentially expressed (DE) genes were identified using DeSeq2 R package (Love et al, 2014) and a FDR corrected p-value <0.05.

### Quantitative Real-Time PCR

### Materials and Methods

### Test Item

| | |
|---|---|
| Identification | ITF3756 |
| Lot/Batch Number | 9 |
| Purity | >95% |
| Molecular Formula | C₁₃H₁₁N₅O₂S |
| Molecular Weight | 301.32 g/mol |
| Expiry/Retest date | November 2021 |
| Storage Conditions | −20° C. |
| Source and Manufacturer | Italfarmaco SpA |
| Special Handling Precautions | Usual protection of all personnel |
| | conducting the study |
| | (mask, gloves and eyeglasses) |

### Test Item Working Solution

Test item has been dissolved in DMSO and diluted into the appropriate medium to the final concentrations needed.

### Experimental Design

To extend the feasibility of the present invention, the potential of ITF3756 to modulate mRNA expression of 17 genes was also tested in PBMC. The gene expression has been determined by comparing the expression of the above genes in the treated cells versus vehicle-treated cells.

PBMC Treatment with Test Item

PBMC were resuspended in RPMI 1640 medium (Dutch modified, ThermoFisher) at the density of 1×106 cells/ml. Cells were plated in 6-well plates (Corning), 3 ml per well. Cells were treated with ITF3576 at four concentrations: 0.25, 0.5, 1 and 2 μM. Each treatment concentration was replicated in 3 wells for experimental triplicate test. Vehicle (DMSO 0.05%) treated cells were plated at the same density and volumes. Plates were incubated for 4 hours at 37° C. 5% C02. At the end of treatment incubation cells were collected for total RNA extraction.

### RNA Extraction and Retro-Transcription

After treatment with the test compound, PBMC were withdrawn from 6-well plates and collected in 15 ml tubes (Falcon). Wells were washed once with 1 ml of PBS pH 7.4 (ThermoFisher) and the wash volume was added to the collected cell suspensions. Cell suspension was centrifuged for 10 minutes at 1500 rpm. Supernatant was discarded and 350 μl lysis buffer from RNeasy Mini kit (Qiagen) was added to cell pellets. Lysed cells were pipetted up and down 10 times to disperse cells debris. RNA extraction procedure was then carried out as for RNeasy Mini kit protocol including the DNase step.

RNA concentration was determined using Nano Drop 1000 spectrophotometer. RNA was diluted with nuclease-free water (Ambion) at 50 ng/ul in a volume of 16 μl. Superscript VILO IV reagent (Invitrogen) were added to RNA as shown below.

TABLE 1
Reagents added to the wells of a 96 well-plate for amplification

| | Volume/well (μL) |
|---|---|
| Superscript VILO IV | 4 |
| CDNA (50 ng/μL) | 16 |
| Total volume | 20 |

The reverse transcription reaction was performed in 96-well plates on the instrument iCycler iQ™ (Bio-Rad) with the following thermal cycle: 10 min at 25° C., 10 min at 50° C. and 5 min at 85° C. 20 μl of resulting cDNA was then diluted with 40 μl of TE buffer (Invitrogen) to a final theoretical concentration of 13.3 ng/μl.

### Gene Expression Detection

The TaqMan 20× Gene Expression Assay reagents (Applied Biosystems) were used for the detection of gene transcripts and are shown in Table 2.

TABLE 2
Features of the TaqMan assays used for qPCR

| | Assay | Span | Amplicon lenght |
|---|---|---|---|
| | name | exon | (bp) |
| Gene name | | | |
| NBEAL2 | Hs01035331_g1 | Yes | 73 |
| SLC27A1 | Hs01587911_m1 | Yes | 120 |
| LTBP4 | Hs00943217_m1 | Yes | 61 |
| ANXA6 | Hs01049082_m1 | Yes | 68 |
| IRF6 | Hs00196213_m1 | Yes | 93 |
| STAB1 | Hs00248439_m1 | Yes | 79 |
| CD84 | Hs01547121_m1 | Yes | 75 |
| CD276/B7-H3 | Hs00987207_m1 | Yes | 65 |
| RANK/TNFRSF11A | Hs00921372_m1 | Yes | 75 |
| CXCL2 | Hs00601974_m1 | Yes | 100 |
| CXCL3 | Hs00171061_m1 | Yes | 99 |
| PD-L1/CD274 | Hs00204257-m1 | Yes | 77 |
| CD206/MRC1 | Hs02832367_g1 | Yes | 81 |
| CD204/MSR1 | Hs00234007_m1 | Yes | 63 |
| CD163 | Hs00174705_m1 | Yes | 72 |
| ADA | Hs01110947_m1 | Yes | 76 |
| MMP-9 | Hs00957555_m1 | Yes | 79 |
| Housekeeping genes | | | |
| UBC | Hs00824723_m1 | Yes | 71 |
| B2M | Hs99999907_m1 | Yes | 75 |
| HPRT1 | Hs99999909_m1 | Yes | 100 |

Real-time PCR was performed employing a CFX C1000TM touch thermal cycler connected to the CFX 96 Touch Real time PCR detector system (Bio-Rad). The qPCR reaction was performed in a 96 well plate (Hard Shell PCR plates Bio-Rad) with Universal Master Mix reagent (Applied) containing AmpliTaq Gold® DNA Polymerase. 3 μl of cDNA corresponding to 40 ng of template were added to PCR reaction reagent for a total volume of 15 μl as shown below:

Automatic excel reports reporting threshold cycles (Ct) values were exported for raw data collection and subsequently elaborated for the calculation of expression modulation.

### Data Acquisition

The level of mRNA expression modulation was evaluated comparing the mRNA level in treated versus not-treated samples; all data were normalized versus the average expression signal of three housekeeping genes (reference genes: UBC, B2M and HPRT1) in the corresponding samples.

To determine the modulation potential of the test item, the “2^{-ΔΔCT}” method was used.
2^{-ΔΔCT} was calculated for each treatment condition and each target as single replicates
ΔΔCt=−(ΔCt, treated−ΔCt, non-treated)
ΔCt, treated=Ct TARGET treated−Ct REFERENCE treated
ΔCt, non-treated=Ct TARGET non-treated−Ct REFERENCE non-treated
Ct TARGET non-treated is intended as the average of three non-treated replicates
Ct REFERENCE treated is intended as the average values of the single replicate over the three housekeeping genes
Ct REFERENCE non-treated is intended as the average of Ct values for the three housekeeping genes over all non-treated replicates.

For treated samples showing 2^{-ΔΔCT} values lower than 1 (down-modulated), the fold modulation value was reported as the ratio between non-treated control 2^{-ΔΔCT} value (corresponding to 1, no modulation) and treated samples 2^{-ΔΔCT} value, adding a minus sign to outline a down-modulation.

With 2^{-ΔΔCT} values upper than 1 there is up-modulation. The fold modulation value was reported as the ratio between non-treated control 2^{-ΔΔCT} value (corresponding to 1, no modulation) and treated samples 2^{-ΔΔCT} value.

### Computer System

The computer systems used on this study to acquire and quantify data included the following systems:

### System Name Function

Spylog v 1.1 Electronic temperature monitoring system for refrigerators and freezers. Supplier: AHSI.
CFX 96 Touch Real time PCR detector system. Supplier: Bio-Rad Laboratories
NanoDrop 1000 Spectrophotometer. Supplier: ThermoFisher iCycler iQ™ Thermo cycler. Supplier: Bio-Rad Laboratories

### Statistical Analysis

All the data were given as means±standard deviation and were the average of experimental triplicates. Statistical evaluation among groups (treated samples vs not-treated samples) were carried out using two-tailed Student's t-test.

In the graphs, the threshold line corresponds to deviation from vehicle-treated control (1) of +/−0.7 (0.7 represent 3 time the standard deviation (SD) of fold change for housekeeping genes over all samples).
** Pvalue <0.05, * Pvalue <0.1

### 5. REFERENCES

1. Analysis of Relative Gene Expression Data Using Real-Time Quantitative PCR and the 2^{-ΔΔCT} Method. K J Livak et al, Methods 25, 402-408 (2001)

### Results

### RNAseq

After 4 hours of incubation of monocytes, the RNAseq analysis shows that hundreds of differentially expressed genes with padj<0.05 were identified in samples treated with ITF3756, the treatments with TNF-α and the combination of ITF3756+TNF-α may not give rise to a modulation with a padj<0.05.

Table 3 shows the number of up- and down-modulated genes for each treatment versus vehicle-treated control (don) in the indicated groups.

TABLE 3
Number of genes modulated according to treatment of monocytes

| | Genes Up regulated | Genes Down regulated |
|---|---|---|
| ITF3756/don | 1499 | 1503 |
| ITF3756 TNFα/don | 2282 | 2329 |
| TNFα/don | 1563 | 1688 |

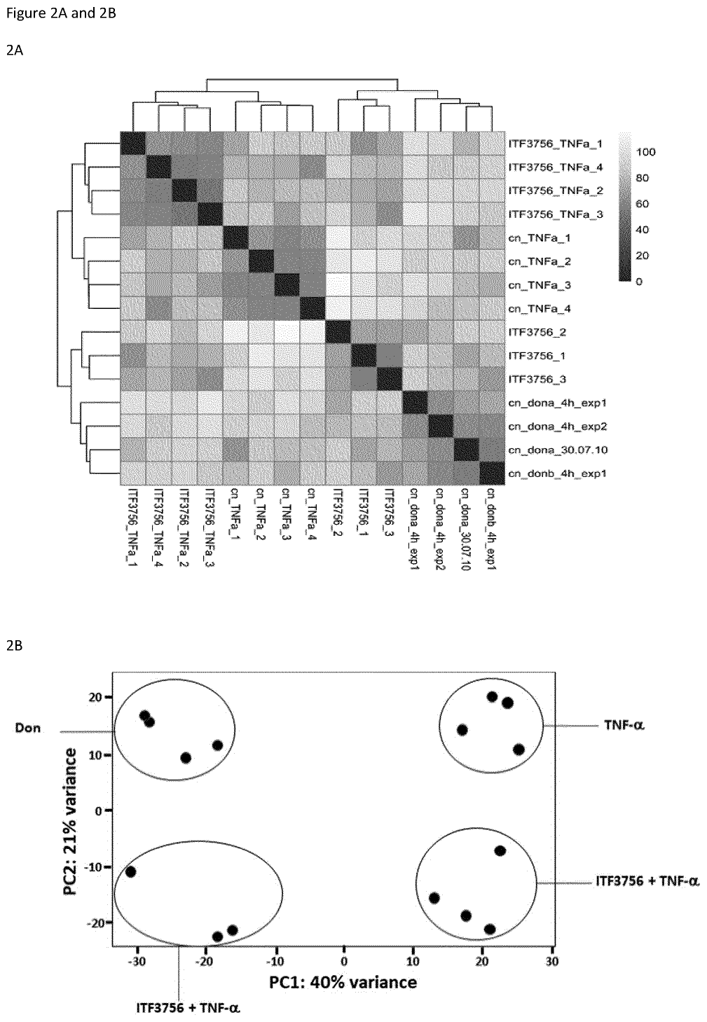

Sample to sample distance and principal component analyses (FIGS. 2 A and 2B, respectively) showed that four distinct population can be identified according to the treatment. This indicates clear different gene expression in the different populations and clustering of samples according to treatment. Overall, both analyses indicate a homogeneous response to the different treatments. This is remarkable since a different response is expected from different donors.

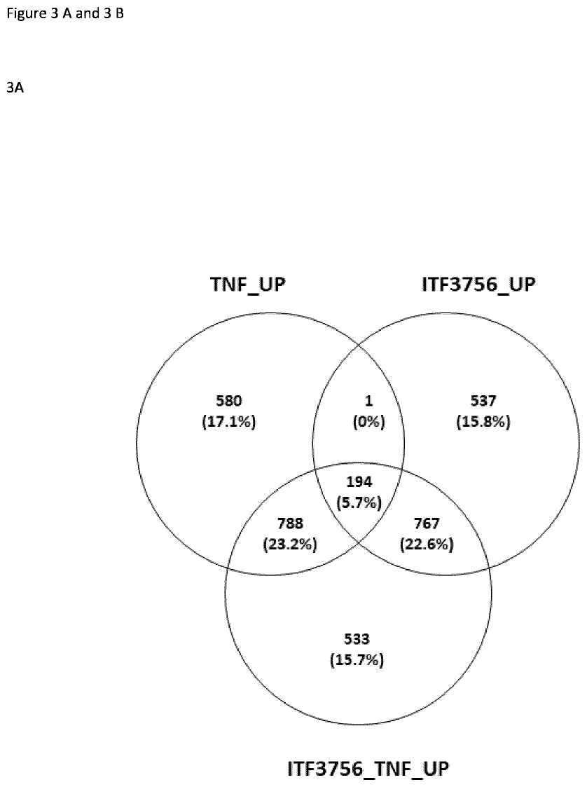

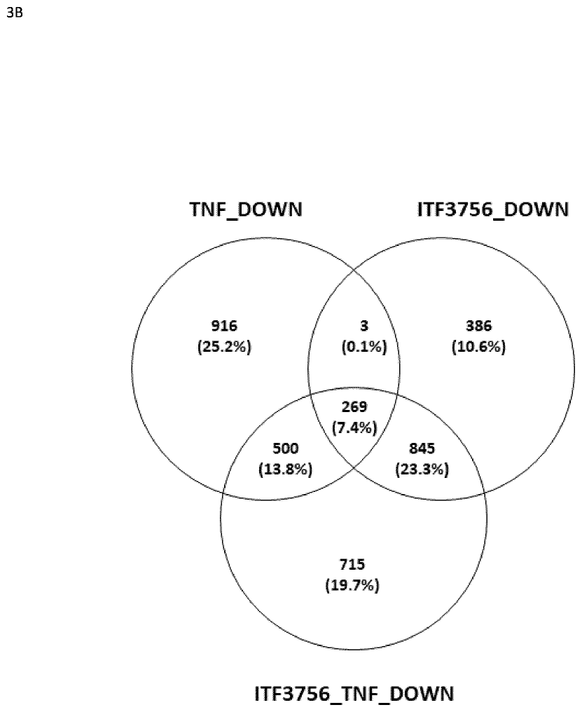

We then identify the genes that were selectively up- or down-modulated by TNF-α, ITF3756 and ITF3756+TNF-α. FIGS. 3A and B show the Venn diagrams for genes upregulated or downregulated versus control, respectively.

The diagrams indicate that there are 537 genes specifically upregulated by ITF3756 and 386 gene specifically downregulated. We were particularly interested in these genes since from them, a specific signature for ITF3756 could be identified.

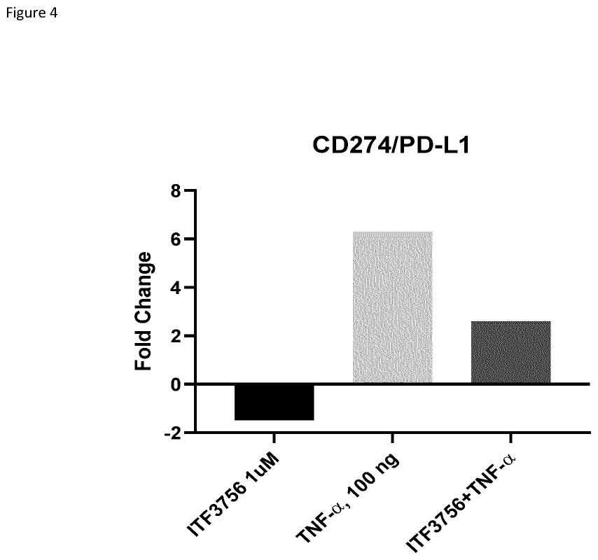

We first analyzed the expression of PD-L1 (CD274) to verify its upregulation induced by TNF-α and its inhibition by ITF3756. FIG. 4 shows that ITF3756 induced a strong downregulation (59%) of PD-L1 gene expression in cells treated with TNF-α+ITF3756.

We then searched for genes that can have an impact on the biological activity of ITF3756 among the genes that were up- or down-modulated. Table 4 summarizes the genes that we selected.

TABLE 4
Selected up- and down-modulated genes with a possible
impact on the biological activity of ITF3756.

| Upregulated | Downregulated | Downregulated upon |
|---|---|---|
| genes | genes | TNF-α |
| NBEAL2 | CD84 | CD274 (PD-L1) |
| SLC27A1 | CD276 | |
| LTBP4 | RANK/TNFRSF11a | |
| ANXA6 | CXCL2 | |
| IRF6 | CXCL3 | |
| CD40 | STAB1 | |

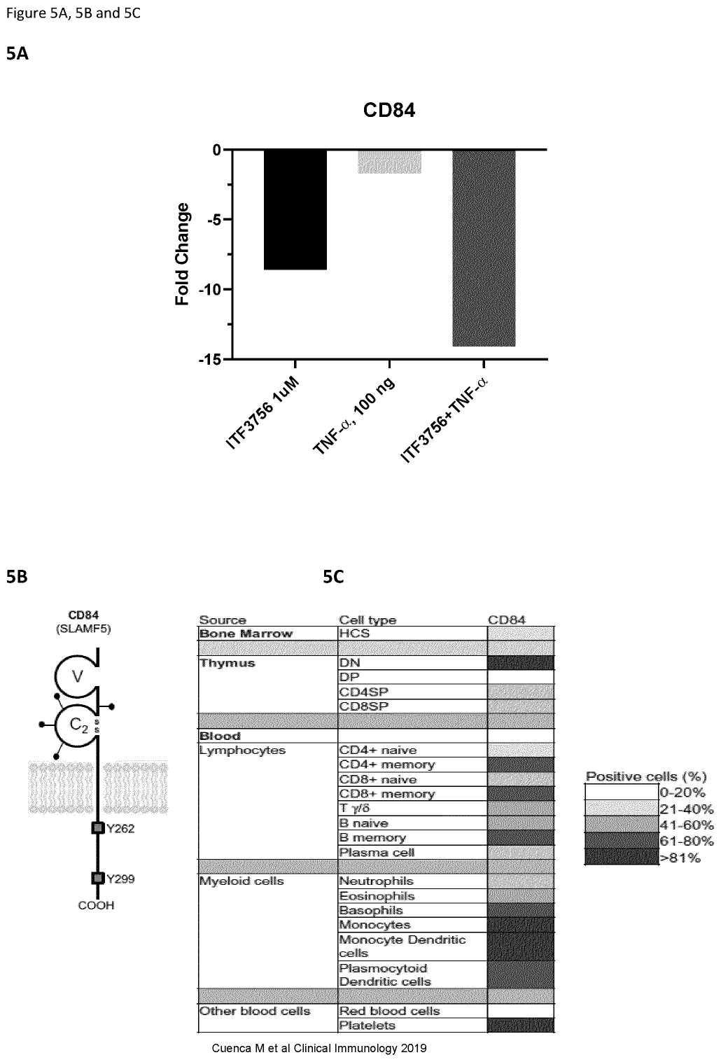

Our experiments clearly demonstrates that ITF3756 strongly downregulates the expression of CD84 as shown in FIG. 5A. Human monocytes treated with ITF3756 1 μM downregulate the expression of CD84 and this downregulation is further enhanced in the presence of a pro-inflammatory stimulus such as TNF-α. FIGS. 5B and C show the schematic structure and immune cells expression of CD84, respectively.

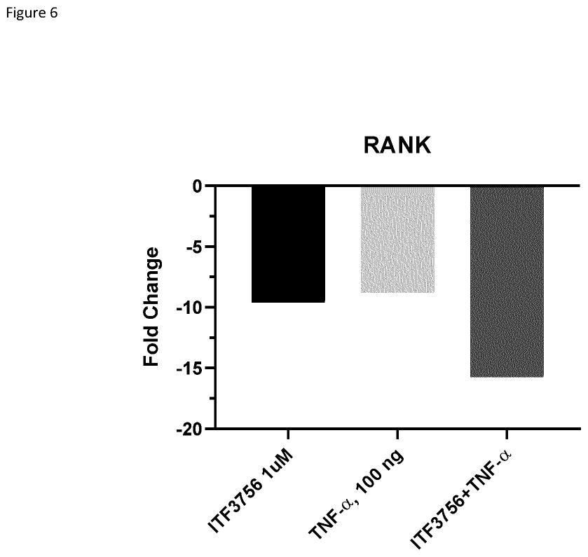

RANK (TNFRSF11A) and RANKL (TNFRSF11) are members of the TNF-receptor superfamily. RANK can be expressed on a variety of cell types including cancer cells, epithelial cells and macrophages in the tumor microenvironment. The interaction with RANKL leads to proliferation and cell migration of tumor cells, angiogenesis and macrophage recruitment and M2 differentiation. ITF3756 induces a strong downregulation of RANK (see FIG. 6) on monocytes suggesting a possible inhibition of M2/pro-tumor macrophage differentiation. This is in agreement with our data that indicates an increase in M1 macrophages in in vitro differentiation in the presence of ITF3756. It can also be observed that, when the cells are treated with TNF-α and ITF3756, the downregulation of RANK/TNFRSF11a is increased compared to single treatments.

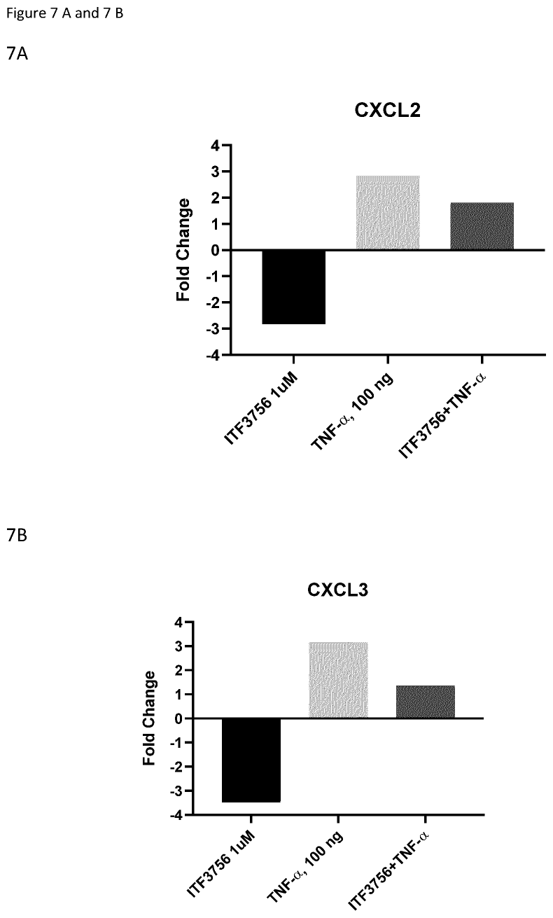

Chemokines are a family of chemoattractant cytokines which play a crucial role in cell migration through venules from blood into tissue and vice versa. CXCL2 and CXCL3 are two chemokines involved in the recruitment and generation of monocytic MDSC and their inhibition has been proposed to decrease mo-MDSC generation and improve host immune-surveillance (Shi et al., 2018). As shown in FIG. 7A, ITF3756 decreases the expression of CXCL2 and reverts the upregulation induce by TNF establishing a normal level of expression.

CXCL3 is another chemokine that affects the differentiation and function of human monocyte-derived dendritic cells, pushing them towards a myeloid-derived suppressor cell (MDSC)-like phenotype. Furthermore, MDSC themselves express CXCR2 receptor that can be activated by CXCL3 promoting their migration to the tumor microenvironment as described in KRAS-mutated colorectal cancer (Liao et al., (2019) Cancer Cell 35:559-572). As shown in FIG. 7B, ITF3756 reduced the expression of this chemokine CXCL3.

The activity on the two chemokines, together with the effect on RANK expression, suggests that ITF3756 downregulates the expression of genes that are related to phenotype of suppressive myeloid cells.

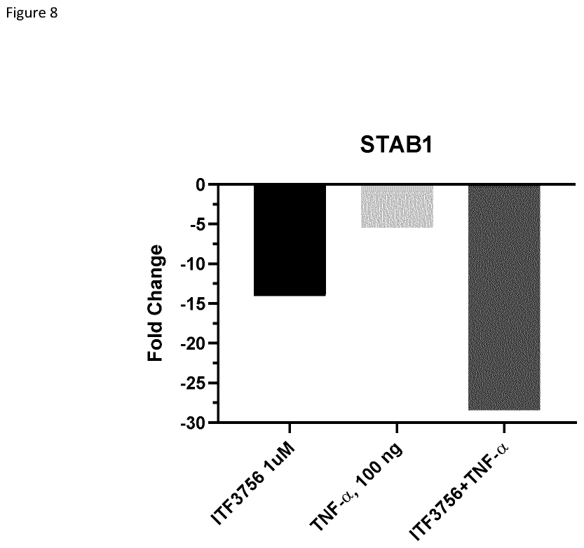

STAB1 also known as Clever-1/Stabilin-1, is another important gene related to a phenotypic change in macrophages and monocytes from immunosuppressive to pro-inflammatory phenotype. STAB1 is strongly downregulated by ITF3756 and synergistically reduced in the presence of TNF-α (FIG. 8).

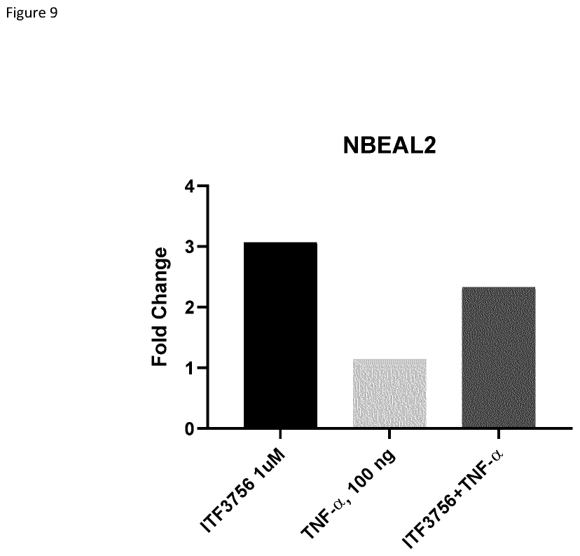

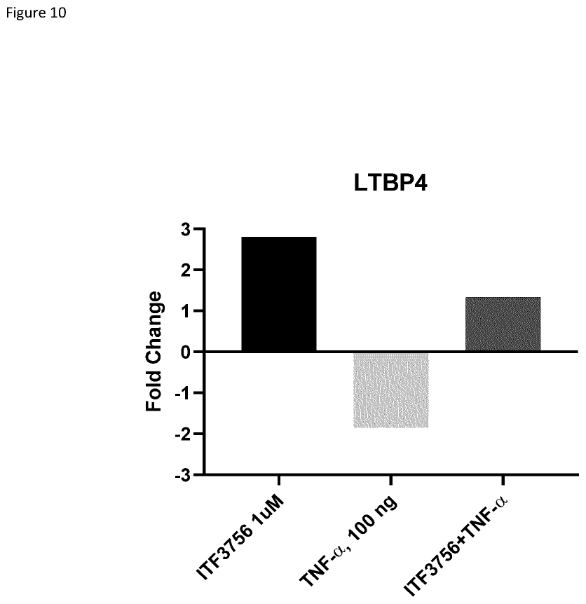

Among the genes that are upregulated by ITF3756, we have selected NBEAL2 (FIG. 9), that is a BEACH-domain-containing protein linked to granule development and LTBP4, that is a key regulator of transforming growth factor beta (TGFB1, TGFB2 and TGFB3) that controls TGF-a activation by maintaining it in a latent state during storage in extracellular space. The LTBP4 gene is downregulated by two fold by TNF-α (see FIG. 10).

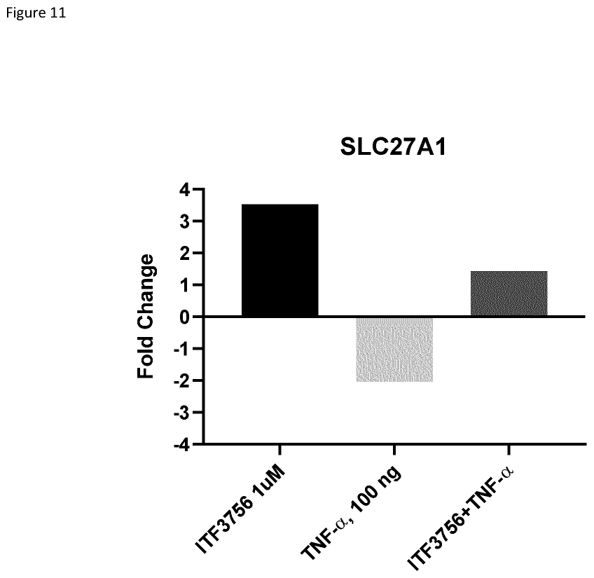

Our results demonstrate that ITF3756 counters this downregulation, but the effect is to restore the normal level of control without further upregulation. FIG. 11 shows the effect of ITF3756 on the expression of FATP1/SLC27A1. This gene is involved in the pro-inflammatory response in macrophages (Nishiyama et al. (2018) International Immunopharmacology 55, 205-215) thus making it a gene involved in the possible pro-inflammatory, anti-tumor response mediated by macrophages. ITF3756 has an opposite effect on this gene compared to TNF-α and when cells are treated with the combination of the two, the basal expression of the gene is maintained.

Another gene with a possible role in mediating a pro-inflammatory effect in myeloid cells is interferon regulatory factor 6 (IRF6), that belongs to a family of nine transcription factors that share a highly conserved helix-turn-helix DNA-binding domain and a less conserved protein-binding domain. Most IRFs regulate the expression of interferon after viral infection. Other IRF members are modulated by ITF3756, but the stronger change of gene expression is exerted on IRF6.

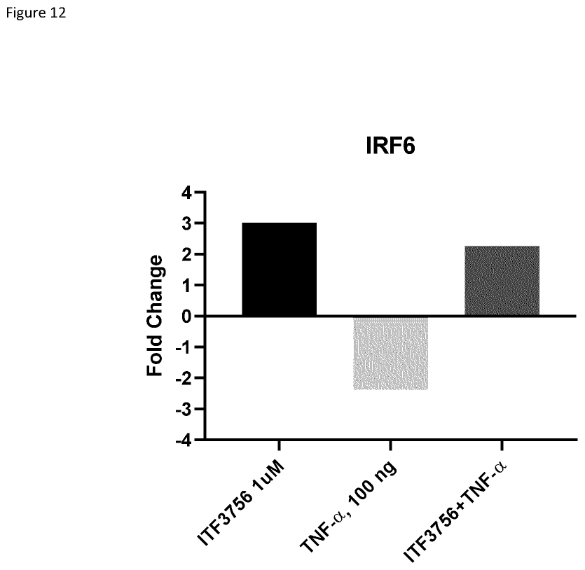

FIG. 12 shows that only ITF3756 induced a modulation of IRF6 expression while TNF-α or the combination had no effect.

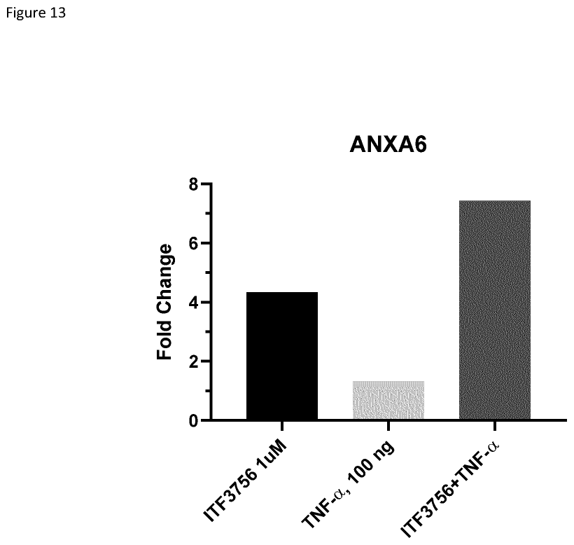

Annexin 6 (AnxA6) is another gene showing a robust upregulation upon ITF3756 treatment. In the presence of TNF-α, the upregulation is even stronger and probably synergistic as shown in FIG. 13.

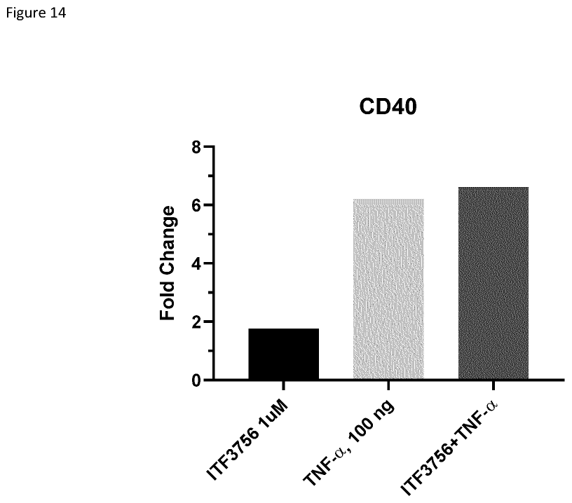

CD40 is a member of the TNF receptor superfamily, it is expressed on a variety of cell types including monocytes/macrophages and dendritic cells. Its engagement by its natural ligand CD40L, leads to T cell activation and induction of anti-tumor macrophages. Activation of the CD40-CD40L axis for the induction of antitumor immune response has been approached in several ways, the more recent being the use of agonistic anti CD40 antibodies. Biological effects and clinical responses have been observed below the MTD. In addition, adverse events appear to be readily manageable in the clinical setting. The induction of CD40 gene expression obtained by ITF3756 (FIG. 14) suggests that it could contribute to the overall antitumor immune stimulation observed with the compound both in vitro and in vivo.

As shown in Table 3, a number of genes were modulated by ITF3756. Some of them have been identified as specific markers of tumor associated macrophages (TAMs). Although this analysis has been conducted on monocytes, the modulation of these genes may have implication for the development of TAMs, that are the major innate immune cells that may constitute a large proportion (up to 50%) of the cell mass of human tumors. TAMs are highly heterogeneous, they may develop from resident, tissue-specific macrophages and from monocytes recruited from the circulation through chemoattractant gradients. Cancer type, stage, and intratumor heterogeneity strongly influence TAM population. The majority of TAMs are programmed by tumor microenvironment to support primary tumor growth and metastatic spread. Nevertheless, tumor microenvironment can influence TAMs to restrict tumor growth and metastasis (Larionova et al., 2020). Tumor promoting macrophages with M2 phenotype express specific markers some of them are robustly downregulated in monocytes treated with ITF3756 as shown in Table 5, supporting the possible implication of the induction of antitumor phenotype once the monocytes are recruited to tumor tissue.

TABLE 5
M2-related genes downregulated by ITF3756 in human monocytes.

| M2 Marker | Fold change |
|---|---|
| CD163 | −4 |
| CD204/MSR1 | −2.6 |
| CD206/MRC1 | −13.3 |
| MMP9 | −3.6 |
| STAB1 (Stabilin- | −14 |
| 1/Clever-1) | |
| ADA | −1.9 |

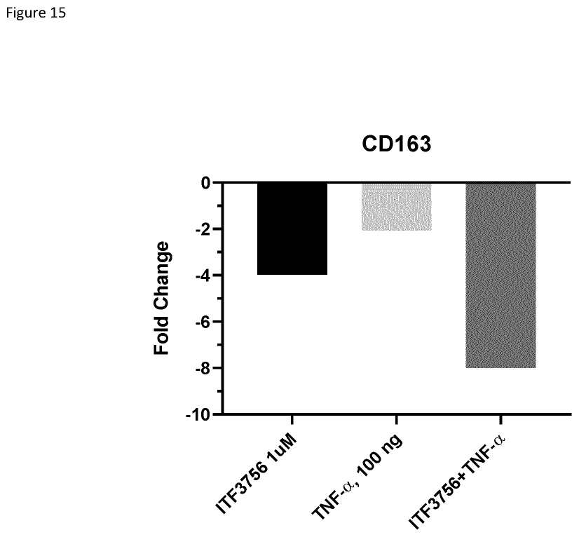

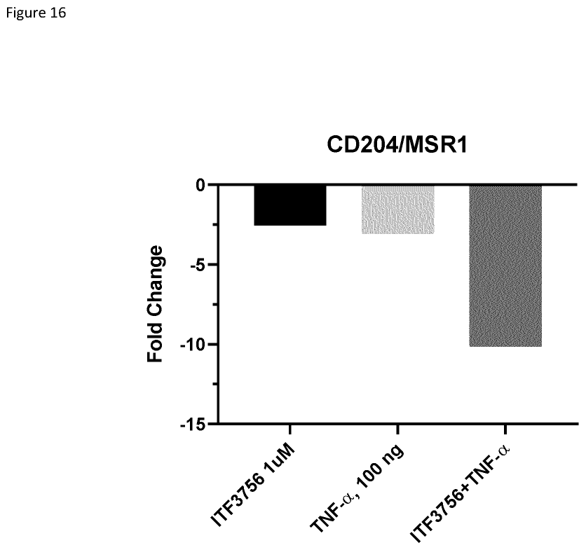

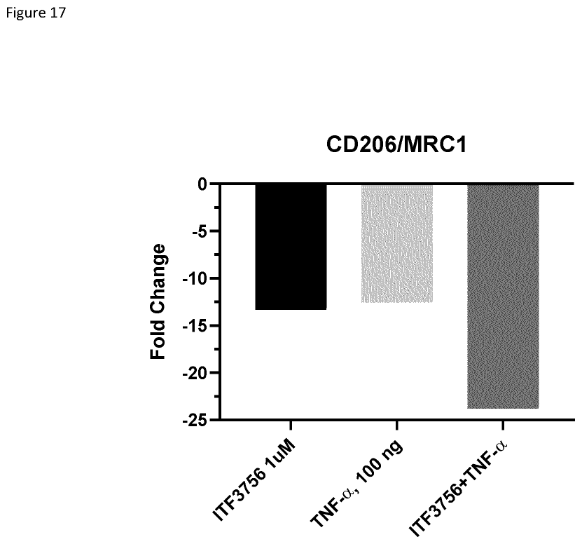

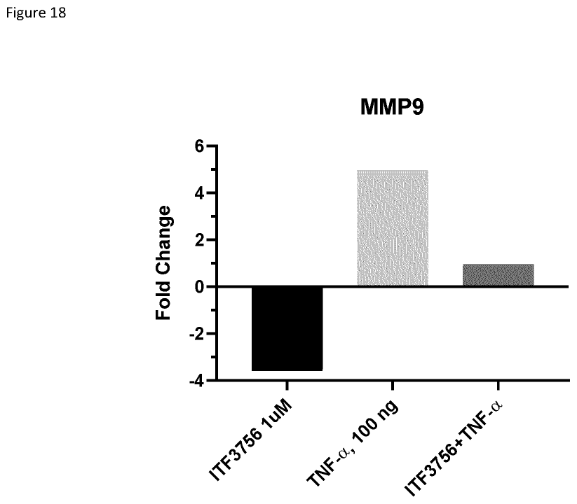

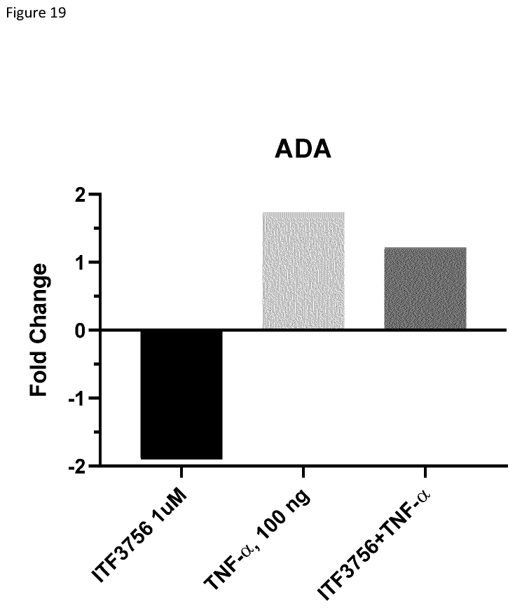

FIGS. 8 (stabilin-1) and FIGS. 15 to 19 show the modulations of the genes in Table 5 including those exerted by TNF-α and the combination TNF-α+ITF3756.

### ITF3756 Exerts its Gene Modulation Activity in PBMC

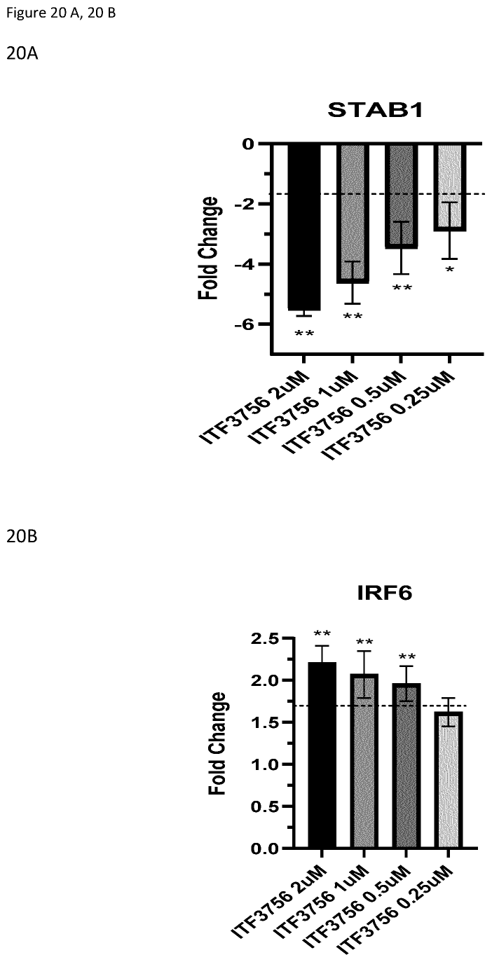

Peripheral blood mononuclear cells can be isolated in a simpler and faster way compared to monocytes. We therefore tested the gene modulation activity of ITF3756 on this cell population using quantitative real time PCR. Results indicates that the results obtained using this approach agree with those obtained with monocytes and RNAseq. FIG. 20 shows the results obtained on two exemplary genes, STAB1 (FIG. 20A) and IRF6 (FIG. 20B) down-modulated and upregulated, respectively, by ITF3756.

Ex-Vivo Gene Expression of Transcripts in Tumor Microenviroment Identified IRF6, MMP9 and CD40 as Biomarkers Associated with Anti-Tumor Response

### Materials and Methods

### Animal Husbandry

### Environmental Acclimation

A minimum acclimation period of 14 days was allowed between animal receipt and the start of treatment in order to accustom the animals to the laboratory environment.

### Housing

Mice were housed inside cages of makrolon (26.7×20.7×h 14 cm) (4-5 mice/cage) with grating cover of steel and bedstead of sawdust of pulverized and sterilized dust-free bedding cobs. Diet and water supply: drinking water were supplied ad libitum. Each mouse was offered daily a complete pellet mouse diet (4RF21, Mucedola) throughout the studies.

### Environmental Conditions

Animals were housed under a light-dark cycle, keeping temperature and humidity constant. Parameters of the animal rooms were assessed as follows: 22±2° C. temperature, 55±10% relative humidity, about 15-20 filtered air changes/hour and 12 hours circadian cycle of artificial light, 7 a.m.-7 p.m.

### Environmental Acclimation

A minimum acclimation period of 14 days was allowed between animal receipt and the start of treatment in order to accustom the animals to the laboratory environment.

ITF3756 (N-hydroxy-4-((5-(thiophen-2-yl)-1Htetrazol1yl)methyl)benzamide).

ITF3756 was synthetized by the Medicinal Chemistry Dept. of Italfarmaco SpA. ITF3756, batch 8, as powder was solubilized in DMSO and stored at −20° C.

### Pharmacological Treatments

TABLE 6
ITF3756 used and pharmacological treatment

| Identification | ITF3756 batch 8 |
|---|---|
| Type of | Solution in the vehicle |
| Formulation | |
| Dose | 5 mg/ml |
| Concentration | |
| Instruction of | Weight an appropriate amount of test |
| Preparation | compound and make a solution at 100 mg/mL |
| | in DMSO. Dilute this solution with H2O/PEG |
| | 1:1 to reach the required dosage of |
| | test article (5 mg/ml solution). |
| | Keep under magnetic stirring. |
| Frequency of | Test formulations was prepared the day of dosing |
| Preparation | |
| Storage Conditions | Room temperature |
| Source and | Italfarmaco SpA |
| Manufacturer | |
| Dosage and Route | 10 mg/kg by intraperitoneal injection |
| Treatment | Once a day from day 10 (Monday) |
| | till day 20. No drug administration |
| schedule | was done on Saturday and Sunday. |
| | Total of 8 administrations |
| Volume | 10 mL/kg (50 mg/kg). Individual |
| | dose volume was calculated based |
| | on the body weight recorded on the day of dosing. |

TABLE 7
Anti PD-1 antibody used and pharmacological treatment

| Identification | Anti mouse PD-1 |
|---|---|
| Lot/Batch Number | 735019O1 and 786520N1B |
| Type of Formulation | Solution in PBS |
| Dosage and Route | 10 mg/kg by intraperitoneal injection |
| Treatment schedule | Alternate day from day 10 (Monday) |
| | till day 20. No drug administration |
| | was done on Saturday and Sunday. Total |
| | of 5 administrations |
| Instruction of | Ready to use solution at 7.7 mg/mL |
| Preparation | (lot. 73501901) or 8.31 |
| | mg/mL (lot. 786520N1B) |
| | Then diluted in PBS at the target |
| | concentrations |
| Frequency of | Test formulations were |
| Preparation | prepared the day of dosing |
| Storage Conditions | +4° C., protected from light |
| Source and Manufacturer | BioXCell |

TABLE 8
Isotype antibody used and pharmacological treatment

| Identification | Rat IgG2b Isotype Control |
|---|---|
| Lot/Batch Number | 707119D1 |
| Type of Formulation | Pharmaceutical Preparation |
| Dosage and Route | 10 mg/kg by intraperitoneal injection |
| Treatment schedule | Alternate day from day 10 (Monday) till |
| | day 20. No drug administration was done on |
| | Saturday and Sunday. Total of 5 administrations |
| Instruction of | Ready to use solution at 9.16 mg/mL. Then |
| Preparation | diluted in PBS at the target concentrations |
| Frequency of | Test formulations were prepared the day of |
| Preparation | dosing |
| Storage Conditions | +4° C., protected from light |
| Source and | BioXCell |
| Manufacturer | |

### Study Design

Adult BALB/c mice were injected s.c. with 1×10⁶ CT26 tumor cells (diluted to 100 μl with phosphate buffered saline) and treated with anti-PD1 or ITF3756 when the tumor volume reached 75-100 mm³, to explore the modulation of genes in the tumor microenvironment by RNAseq. Since ITF3756 acts on the PD-1/PD-L1 axis, this approach allows the identification of genes specific and common to both treatments. The overall schedule is described in Table 9.

TABLE 9
Overall schedule of treatments

| | | Injection | | | |
|---|---|---|---|---|---|
| | | s.c. | | | |
| Group | Mice | CT26 | Treatment | Frequency | Note |
| 1 | 5 | + | Untreated | | |
| 2 | 5 | + | ITF3756 | from Mon to Fri + | 50 mpk os; sacrifice 1 h |
| | | | | from Mon to Wed. | post last treatment |
| | | | | 8 administrations | |
| 3 | 5 | + | ITF3756 | Ibidem | 50 mpk os; sacrifice 4 h |
| | | | | | post last treatment |
| 4 | 5 | + | ITF3756 | Ibidem | 50 mpk os; sacrifice 18 h |
| | | | | | post last treatment |
| 5 | 5 | + | ITF3756 | Ibidem | 50 mpk os; sacrifice 24 h |
| | | | | | post last treatment |
| 6 | 5 | + | Vehicle | Ibidem | Sacrifice 4 h post last |
| | | | | | treatment |
| 7 | 5 | + | Anti-PD1 | Mon-Wed-Fri + | 10 mpk ip; sacrifice 1 h |
| | | | | Mon-Wed) | post last treatment |
| | | | | 5 administrations | |
| 8 | 5 | + | Anti-PD1 | Ibidem | 10 mpk ip; sacrifice 4 h |
| | | | | | post last treatment |
| 9 | 5 | + | Anti-PD1 | Ibidem | 10 mpk ip; sacrifice 18 h |
| | | | | | post last treatment |
| 10 | 5 | + | Anti-PD1 | Ibidem | 10 mpk ip; sacrifice 24 h |
| | | | | | post last treatment |
| 11 | 5 | + | Isotype AB | Ibidem | 10 mpk ip; sacrifice 4 h |
| | | | | | post last treatment |

### Immunoblotting Analysis

Whole cell extracts were obtained by lysing mice spleens with Triton Buffer (50Mm Tris-HCl ph 7.5, 250 mM NaCl, 50 mM NaF, 1 mM EDTA pH 8, 0.1% Triton), supplemented with protease and phosphatase inhibitors (Roche, Germany). Proteins were separated by SDS-PAGE, transferred onto PVDF membranes and blocked with PBS-T (Phosphate-buffared saline and 0.1% Tween-20 containing 5% non-fat dry milk for one hour at room temperature (RT). The incubation with primary antibodies was performed for two hours at RT, followed by incubation with the appropriate horseradish peroxidase-conjugated secondary antibody. Detection was performed with ECL Western Blot Reagent (Amerscham). The antibodies used were: mouse anti acetylated tubulin (Sigma, T6793), mouse anti tubulin (Sigma, T6074), goat anti-mouse IgG (H+L)-HRP conjugate (Bio-Rad, 1706516).

Densitometry analysis was performed using ImageJ software. RNA

### RNAseq

RNA was extracted from flash frozen tumors using the Qiagen extraction kit and stored at −80° C. A paired-end sequencing was chosen, in which short reads are obtained from ends of DNA fragments for ultra-high-throughput sequencing. Prior to further analysis, a quality check was performed on the sequencing data. All samples contain sequences 75 nucleotides long (75 nt×2).

The RNA-Seq analysis pipeline involved several steps:
1. Quality Control of the reads,
2. Removal of low quality reads
3. Reads spliced Alignment
4. Transcriptome expression quantification analysis

### Quality Control of the Reads

The Quality Control is the method used to checks on the quality of the raw data sequencing based on statistics and returning graphs and tables that provide information about the areas where problems may occur.

To perform this step we used the tool for high throughput sequence data namely FastQC tool available on http://www.bioinformatics.babraham.ac.uk/projects/fastqc. FastQC tool return an html report in which you can visualizes the information about your raw data sequencing. The calculation of the Quality Value is performed based on the history “phred score”.

The quality value of the phred score (q) uses a mathematical scale to convert to a logarithmic scale the estimated probability for the incorrect identification of a base (s):

q=−10*log 10(s).

The probability of identifying a base incorrectly equal to 0.1 (10%), 0.01 (1%) and 0.001 (0.1%) produce, respectively, a value of phred score (q or Q) of 10, 20 and 30.

FastQC tool gives a Summary judgment (pass (green symbol), warn (orange Symbol), fail (red symbol)). The phred score is returned in the in the “Per Base Sequence Quality” module of QC report.

### Removal of Low Quality Reads

The NGSQCTool kit tool was employed in order to filter out reads with low Phred quality scores.

### Reads Alignment

The sample was mapped on reference Mus musculus genome (mm10) [4] using the bioinformatics tool STAR (version 2.4.0d), with the standard parameters for paired reads. The reference track was the assembly mm10 obtained from Refseq. The table below shows the percentage of mapped reads for each sample. Average mapping ratio was above 93% and Ribosomal content was below 1% for all samples.

### Transcriptome Expression Quantification

The quantification of transcripts expressed for each sequenced sample was performed using Cufflinks. The units of measurement used in Cufflinks is FPKM (Fragments Per Kilobase of transcript per Million mapped reads) and it is meant to be a measure of relative abundance of a transcript/gene in a RNA pool. It is not intended to be used directly for Differential Expression but it is meant to be human readable, and takes into account main technical confounding factors such as millions of reads and gene length.

### Identification of Responders and Non-Responders

Responders and non-responders were selected based on tumor volume at day 20. The volume of the tumor at the beginning of the treatments (day 10) was on average about 75 mm³. Based on this value of tumor volume, we classified the animals according to the following criteria:
Animals with tumor volume lower or equal to 75 mm³ were considered RESPONDERS (R)
Animals with tumor volume greater than 75 mm³ were considered NON RESPONDERS (NR)

To identify those genes that can be associated with R and NR regardless of treatment, all R and NR (including untreated animals) were taken into account.

To identify specific genes modulated by ITF3756, all animals in this group and in its corresponding vehicle control group were considered for the identification of a possible correlation with tumor response.

To identify specific genes modulated by anti-PD-1 treatment, all animals in this group and in the isotype control group were considered for the identification of a possible correlation with tumor response.

Total counts from RNAseq data were used to compare the gene expression between R and NR.

Statistical significance was determined by unpaired t-test using GraphPad software (version 9), p-values 50.05 were considered significant.

### Results

### ITF3756 Target Engagement: Increased Tubulin Acetylation.

To ascertain HDAC6 inhibition by ITF3756, the spleen of the animals were collected at all sacrifice time points (1 hour, 4 hours, 18 hours and 24 hours). Tubulin acetylation and total tubulin were detected by western blotting.

Tumor bearing animals were sacrificed at various time points after the last administration. Spleens were collected and total splenocytes suspension was prepared. Pelleted cells were lysed to obtain a total protein extract thaw as separated by electrophoresis. Tubulin and acetyl-tubulin were detected after western blotting using specific antibodies.

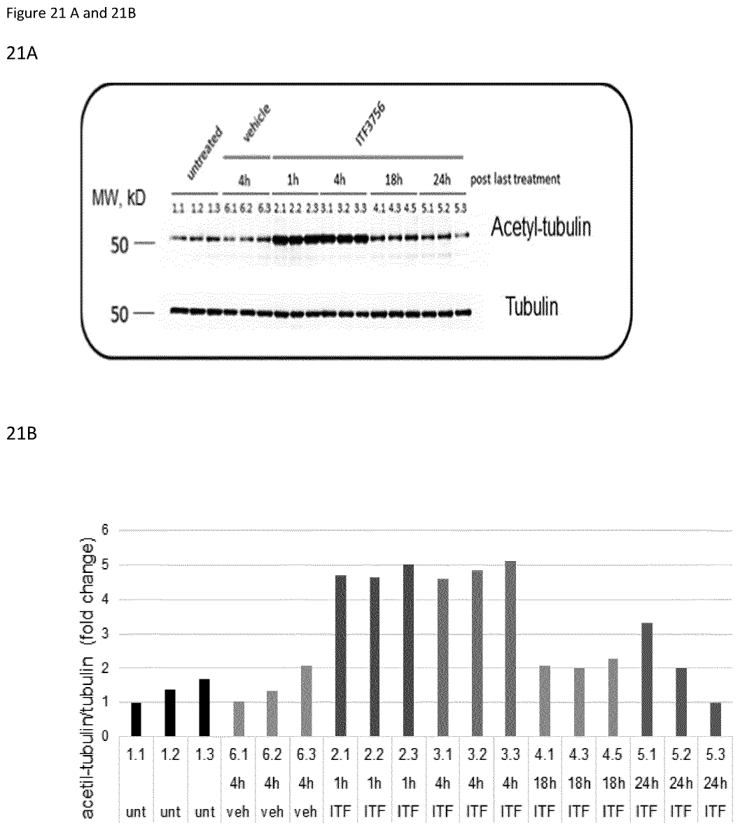

FIG. 21 shows that tubulin acetylation occurred rapidly. One hour after the last treatment, the animals examined had a strong increase that was maintained until 4 hours. Longer wash-out led to a reduction of tubulin acetylation, but it was still high and consistent after 18 hours. Results obtained after 24 hours of wash-out, indicates that the acetylation of tubulin is still detectable, but not in all the animals analysed since one animal out of three had basal level of the acetyl-tubulin.

### Anti-PD-1 and ITF3756 Treatments

Anti-PD-1 immunotherapy is particularly subjected to heterogeneous responses as evidenced by both pre-clinical and clinical studies. This heterogeneity is dependent on the single subject response to immune system stimulation. In agreement with the immune-dependent antitumor activity of ITF3756, we found a heterogeneous response similar to anti PD-1 treated animals.

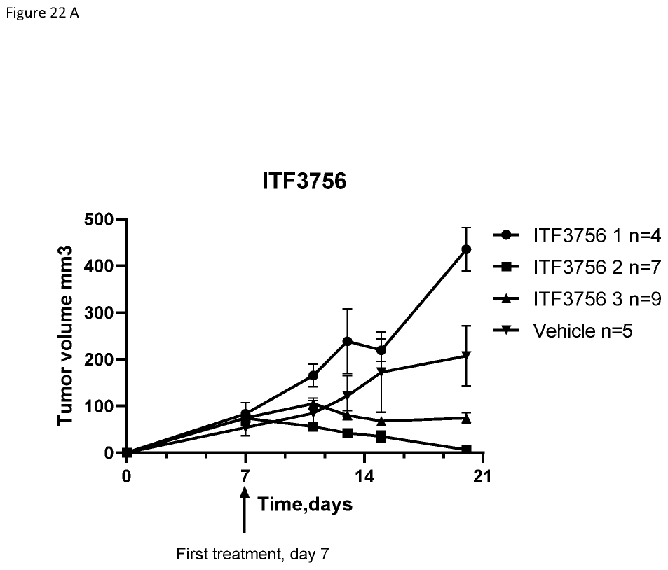

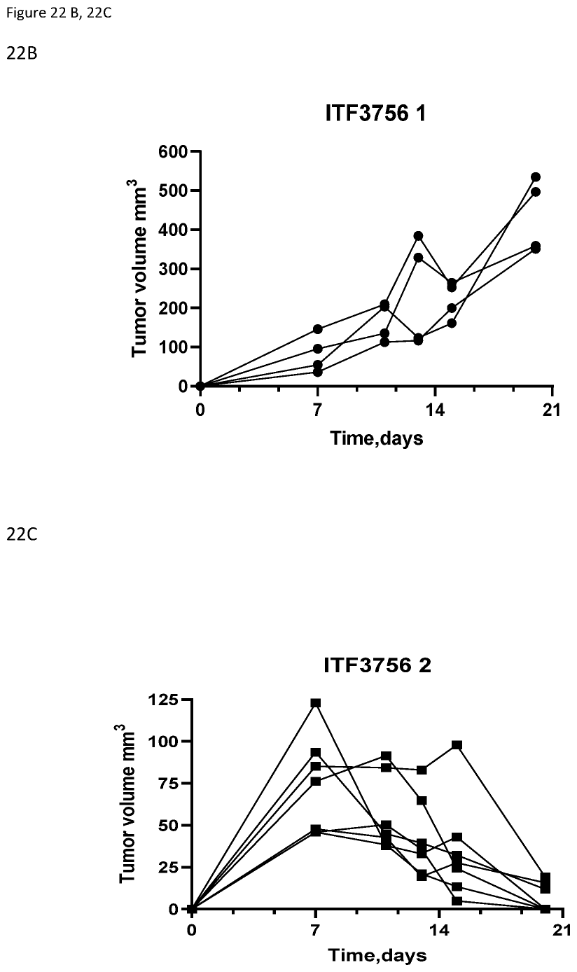

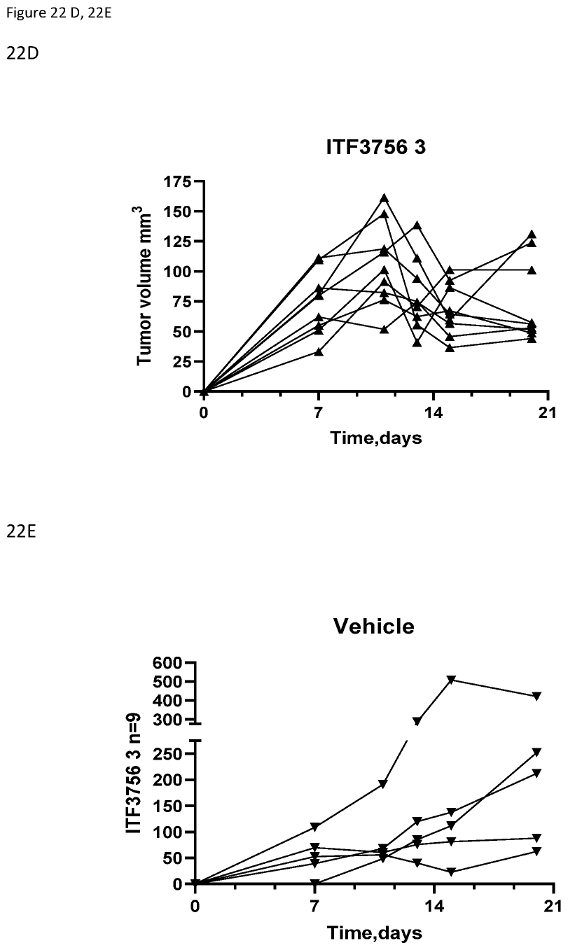

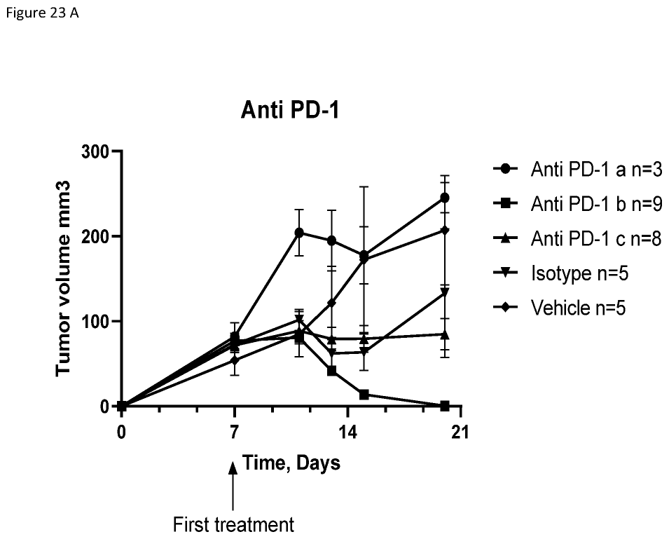

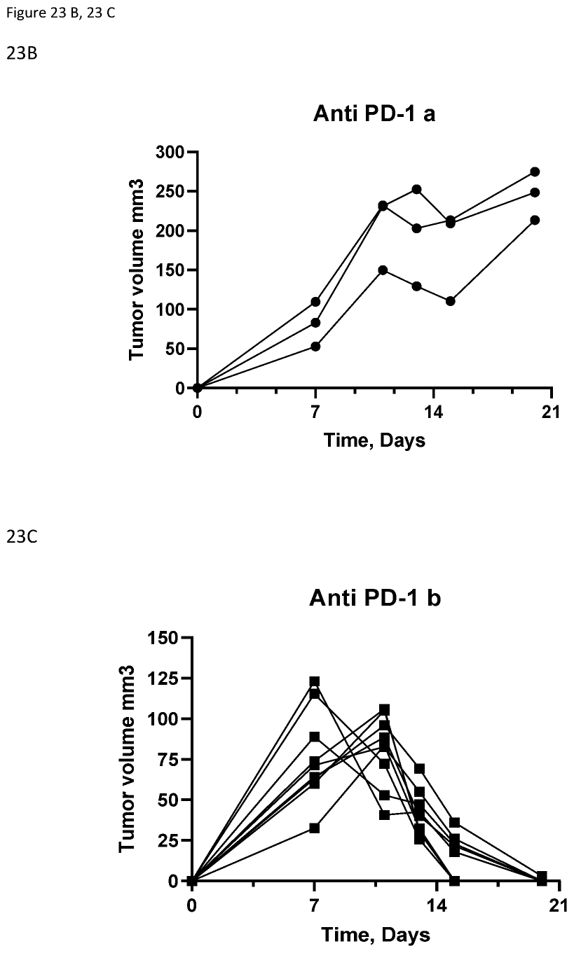

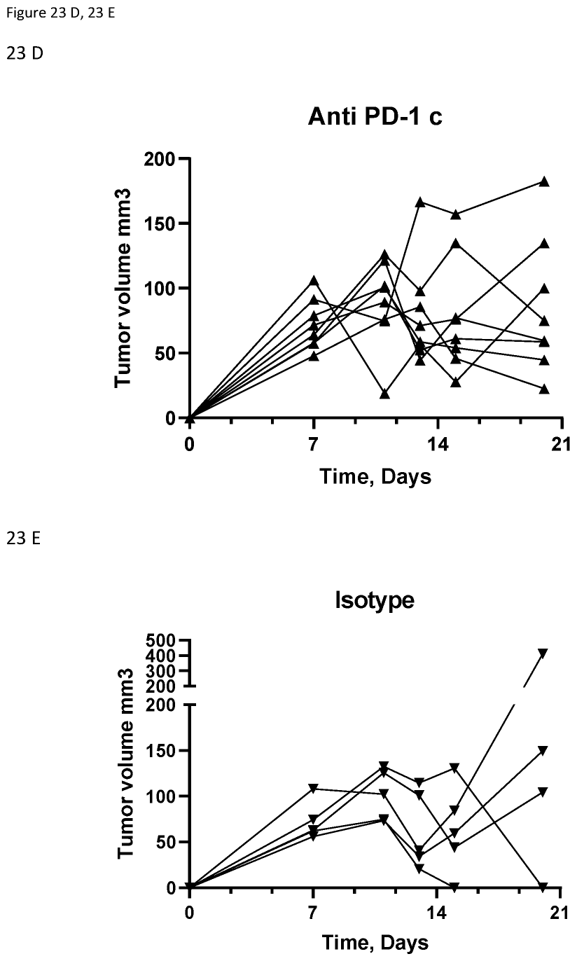

For both treatments, three groups of animals with different tumor reduction could be identified (FIG. 22 and FIG. 23):
animal that did not respond to treatments (ITF3756 1 in FIG. 22; anti PD-1 a in FIG. 23)
animals with tumor strong or complete reduction compared to day 10 (ITF3756 2 in FIG. 22; anti PD-1 b in FIG. 23)
animals with stable tumor mass or slightly above the tumor mass (75 mm³) at the beginning of the treatments (ITF3756 3 in FIG. 22, anti PD-1 c in FIG. 23). For the correlation analysis only the animals in this group having tumor volume lower or equal to 75 mm³ were considered.
   Downregulation of MMP9 and Upregulation of IRF6 and CD40 Correlate with Responders.

Tumor promoting macrophages with M2 phenotype express specific markers some of them are robustly downregulated in monocytes treated with ITF3756 as shown in Table 3. MMP9 is one of these genes and it is therefore associated with a pro-tumorigenic phenotype of macrophages.

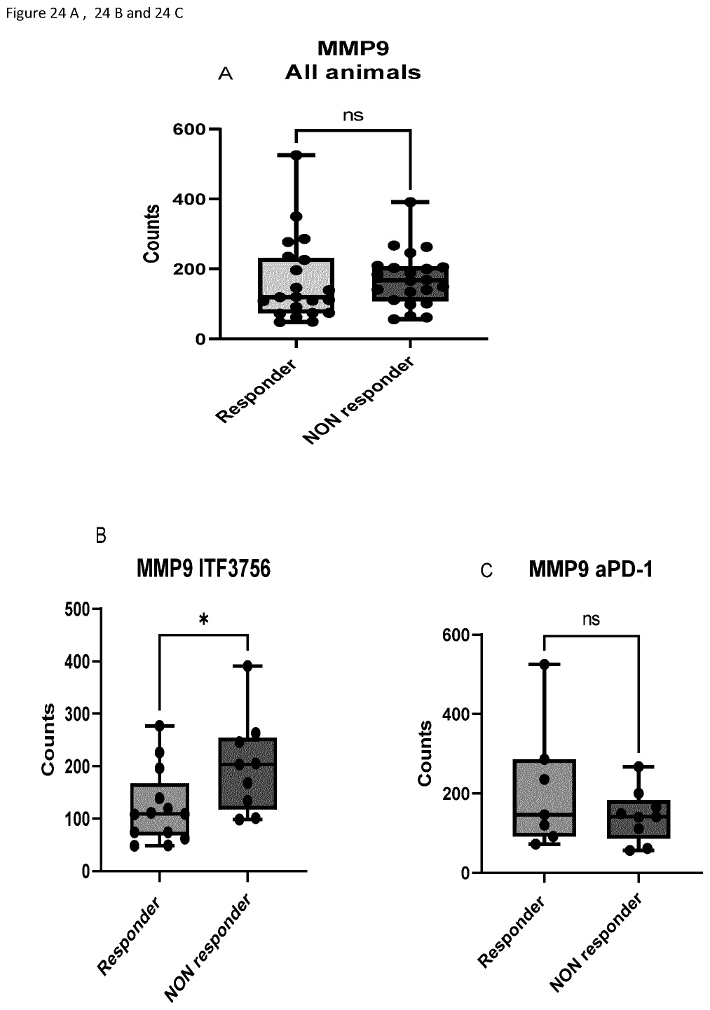

We discovered that the downregulation of MMP9 in the tumor microenvironment of CT26-bearing animals is significantly associated with responder animals of ITF3756 group (FIG. 24). This association does not occur when all animals are considered or when anti PD-1 group and relative control are considered.

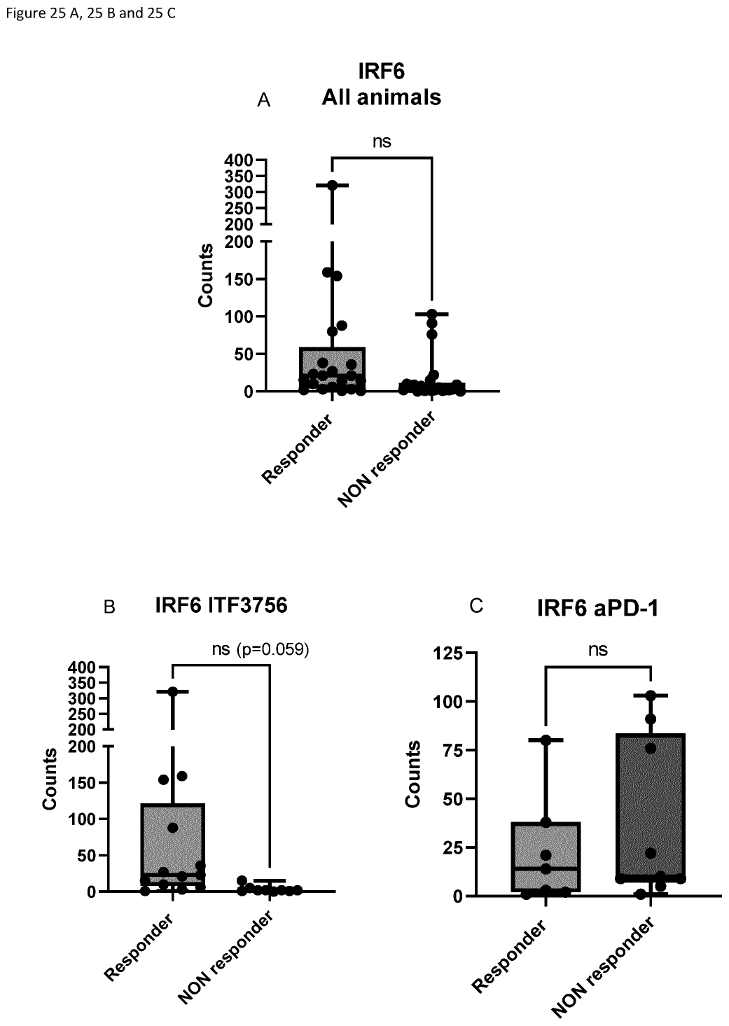

Activation and control of inflammation in the tumor microenvironment is crucial for a proper stimulation of the antitumor immune response. We have identified two genes that are involved in the process of remodelling of the TME from non-inflamed and immune-resistant to inflamed and immune-permissive. One gene is interferon regulatory factor 6 (IRF6). IRF6 belongs to a family of nine transcription factors that share a highly conserved helix-turn-helix DNA-binding domain and a less conserved protein-binding domain. Most IRFs regulate the expression of interferon after viral infection. IRF6 is better known for its association with craniofacial development, but it may have a role in MyD88 signalling together with IRF1 (Honda and Taniguchi, 2006). ITF3756 upregulates the expression of IRF6 in human monocytes and we discovered that its upregulation is associated with responder animals treated with ITF3756 (p<0.1) as shown in FIG. 25. Strikingly, the animal with the highest counts for IRF6 from RNAseq data, had a complete tumor reduction after ITF3756 treatment.

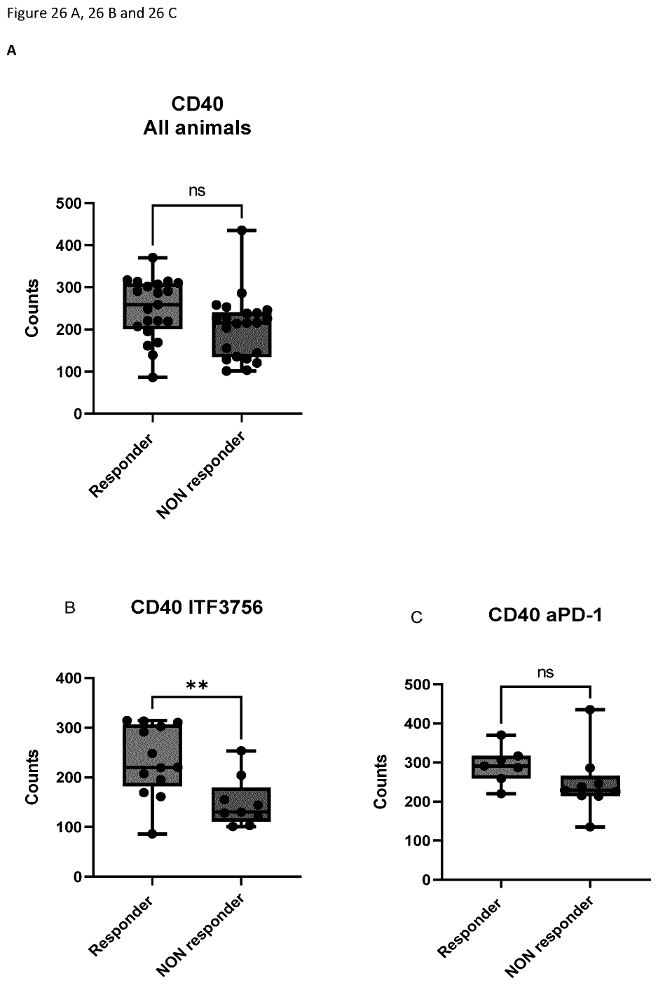

The role of CD40 has been briefly described before. Human monocytes treated with ITF3756 showed an increased gene expression of CD40 which is directly linked to T cell co-stimulation and induction of anti-tumor macrophages. In agreement with this observation, we found that responder mice had a significantly higher gene expression of CD40 (FIG. 26B). Even for CD40, the correlation with responder mice was not found if all animals and animals treated with anti PD-1 antibody were considered (FIG. 26A, C).

1-12. (canceled)

13. A method for evaluating the efficacious dose and/or the biological activity of an HDAC6 inhibitor, comprising steps:
a) determining the expression level of at least one RNA biomarker modulated by an HDAC6 inhibitor and selected from CD84, RANK/TNFRSF11a, CXCL3, CXCL2, STAB1, CD163, CD204/MSR1, CD206/MRC1, MMP9, NBEAL2, LTBP4, ANXA6, FATP1/SLC27a1, ADA, CD276, CD40 or IRF6 genes in a biological sample of a patient; and
b) comparing said expression level with that of a reference sample.

14. The method according to claim 13, wherein the efficacious dose and/or the biological activity of the HDAC6 inhibitor is evaluated during or after the therapeutic treatment of a patient affected by cancer.

15. The method according to claim 13, further comprising step c) classifying the patient as responsive or non-responsive to a therapeutic treatment.

16. The method according to claim 15, wherein the classification of the patient as responsive or non-responsive to the therapeutic treatment is based on whether the expression value of the at least one RNA biomarker is above or below a threshold expression value.

17. The method according to claim 13, wherein the method is an in-vitro or ex-vivo method.

18. The method according to claim 13, wherein the expression level of the RNA biomarkers NBEAL2, LTBP4, ANXA6, FATP1/SCL27a1, CD40 or IRF6 is up-regulated by a HDAC6 inhibitor and the expression level of the RNA biomarkers CD84, RANK/TNFRSF11a, CXCL3, CXCL2, STAB1, CD163, CD204/MSR1, CD206/MRC1, ADA, CD276 or MMP9 is down-regulated by a HDAC6 inhibitor.

19. The method according to claim 13, wherein the expression level of at least the RNA biomarkers STAB1, CD84, CD206/MRC1, MMP9, CD163, CD40 and IRF6 is evaluated.

20. The method according to claim 19, wherein the expression level of at least the RNA biomarkers MMP9, CD40 and IRF6 is evaluated.

21. The method according to claim 13, wherein the HDAC6 inhibitor is selected from tubacin, tubastatin, nexturastat, ACY-1215, ACY-738, ACY-1083, KA2507, T518, SW100 or N-hydroxy-4-((5-(thiophen-2-yl)-1 H-tetrazol-1-yl)methyl) benzamide (ITF3756).

22. The method according to claim 21, wherein HDAC6 inhibitor is N-hydroxy-4-((5-(thiophen-2-yl)-1 H-tetrazol-1-yl)methyl)benzamide.

23. The method according to claim 13, wherein the biological sample is a tissue sample or a body fluid.

24. The method according to claim 23, wherein the tissue sample is a tumor biopsy or blood cells.

25. The method according to claim 23, wherein the body fluid is blood, serum or plasma.

26. The method according to claim 23, wherein the expression level of the at least one RNA biomarker in step a) is detected by RNA sequencing, quantitative RT-PCR, digital PCR, Affymetrix microarray, custom microarray or nanostring technology.

27. The method according to claim 14, wherein the cancer is selected from Adrenocortical Carcinoma, Anal Cancer, Astrocytomas, Basal Cell Carcinoma of the Skin, Bladder Cancer, Brain Tumors, Breast Cancer, Carcinoma of Unknown Primary, Cardiac Tumors, Cervical Cancer, Cholangiocarcinoma, Colorectal Cancer, Endometrial Cancer, Esophageal Cancer, Intraocular Melanoma, Fallopian Tube Cancer, Gallbladder Cancer, Gastric Cancer, Gastrointestinal Carcinoid Tumor, Gastrointestinal Stromal Tumors (GIST), Germ Cell Tumors, Testicular Cancer, Head and Neck Cancer, Hepatocellular Carcinoma, Islet Cell Tumors, Pancreatic Neuroendocrine Tumors, Langerhans Cell Histiocytosis, Leukemias, Lung Cancer (Non-Small Cell, Small Cell, Pleuropulmonary Blastoma, and Tracheobronchial Tumor), Melanoma, Merkel Cell Carcinoma, Mesothelioma, Midline Tract Carcinoma With NUT Gene Changes, Multiple Endocrine Neoplasia Syndromes, Multiple Myeloma/Plasma Cell Neoplasms, Myelodysplastic Syndromes, Myelodysplastic/Myeloproliferative Neoplasms, Neuroblastoma, Ovarian Cancer, Pancreatic Cancer, Paraganglioma, Parathyroid Cancer, Penile Cancer, Pheochromocytoma, Pituitary Tumor, Primary Peritoneal Cancer, Prostate Cancer, Renal Cell Cancer, Retinoblastoma, Sarcomas, Squamous Cell Carcinoma of the Skin, Thymoma and Thymic Carcinoma, Thyroid Cancer, Transitional Cell Cancer of the Renal Pelvis and Ureter, Uterine Cancer, Vaginal Cancer, Vascular Tumors, Vulvar Cancer or Wilms Tumor.

28. The method according to claim 27, wherein the cancer is melanoma, renal cell carcinoma, non small cell lung cancer or colorectal cancer.

29. A kit for use in evaluating the efficacious dose and/or the biological activity of a HDAC6 inhibitor, comprising a multi-well plate and suitable primers and/or probes for determining the expression level of at least one of the RNA biomarkers according to claim 13.
